(19) United States
(12) Patent Application Publication
(10) Pub. No.: US 2026/0226527 A1
(43) Pub. Date: Aug. 6, 2026

(54) METHODS FOR IDENTIFYING HERBICIDAL COMPOUNDS

(71) Applicant: SYNGENTA CROP PROTECTION AG, Basel (CH)

(72) Inventor: Christian Guy NOBLE, Bracknell, Berkshire (GB)

(73) Assignee: SYNGENTA CROP PROTECTION AG, Basel (CH)

(21) Appl. No.: 19/153,019

(22) PCT Filed: Feb. 1, 2024
(86) PCT No.: PCT/EP2024/052542

§ 371 Date: Aug. 1, 2025

(30) Foreign Application Priority Data
Feb. 3, 2023 (EP) 23154964.3
Aug. 16, 2023 (EP) 23191742.8

(51) Int. Cl. C12Q 1/52

(52) U.S. Cl. CPC C12Q 1/52; G01N 2500/04

(57) ABSTRACT

The present invention relates to an in vitro method for determining whether or not a candidate compound is an inhibitor of diaminopelargonic acid aminotransferase (DAPA-AT) said method comprising (i) assaying the candidate compound in an assay mixture comprising (a) the DAPA-AT, (b) an amine donor, (c) pyridoxal 5′-phosphate (PLP) and (d) 7-keto-8-aminopelargoic acid (KAPA), and (ii) detecting inhibition of the DAPA-AT by the candidate compound; characterised in that the amine donor comprises an aliphatic diamine or aliphatic polyamine.

Biotin, also known as vitamin B7, is an essential co-factor for enzymes involved in cellular processes including metabolism of fats, proteins or carbohydrates. Plants and most fungi/bacteria are able to synthesise biotin in contrast to animals which derive biotin from dietary sources or gut bacteria. As such, inhibition of enzymes on the biotin synthetic pathway in plants offer an attractive herbicidal target.

In bacteria, biotin is synthesised from pimeloyl-CoA and alanine via the activity of four enzymes (BioF, BioA, BioD and BioB) which are encoded by genes located in an operon. BioF, also known as 7-keto-8-aminopelargonic acid (KAPA) synthase (EC 2.3.1.47), catalyses the production of KAPA from pimeloyl-CoA and L-alanine. BioA, also known as 7,8-diaminopelargonic acid aminotransferase (DAPA-AT) or DAPA synthase (EC. 2.6.1.62) then carries out the next step, converting KAPA to 7,8-diaminopelargonic acid (DAPA). BioD, also known as dethiobiotin synthetase (DTBS) (EC 6.3.3.3), subsequently converts DAPA to dethiobiotin (DTB) which is in turn converted to biotin via the activity of BioB, also known as biotin synthase (EC 2.8.1.6).

In plants, DAPA-AT is represented by a BioA ortholog B101; and DTBS by a BioD ortholog BIO3. The genes encoding BIO1 and BIO3 are present at a single genetic locus within the plant genome and, in principle, both single and chimeric BIO3-B/01 transcripts can arise through alternative splicing event, potentially giving rise to a bi-cistronic transcript capable of producing separate B103 and BIO1 proteins; or a mono-cistronic transcript giving rise to a bifunctional fusion protein, BIO3-BIO1 also known as BioDA (Dethiobiotin synthetase/7,8-diaminopelargonic acid aminotransferase). However, it is understood that the BIO3-BIO1 fusion protein is the major, if not exclusive, protein produced by the BIO3-BIO1 locus in plants. Thus, the BIO3-BIO1 is a single, bifunctional enzyme that catalyses two independent steps in biotin biosynthesis. Inhibition of the BIO3-BIO1 enzyme, and in particular the BIO1 catalytic domain thus represents an attractive herbicidal target. The present invention thus relates to methods for identifying inhibitors of DAPA-AT.

DAPA-AT is a PLP (pyridoxal 5′-phosphate)-dependent enzyme and several biochemical assays have been developed and reported to examine DAPA-AT activity. Many of these assays have been developed using the bacterial BioA enzyme, however Cobessi et al. (2012) The Plant Cell, Vol. 24: 1608-1625 describe the development of an assay which involves the Arabidopsis BIO3-BIO1 enzyme. However, a common feature of all of these assays reported to date is that they employ S-adenosyl-L-Methionine (SAM) as the amino group donor in the assay, which is converted to S-denosyl-4-methylthio-2-oxobutanoate (Scheme 1).

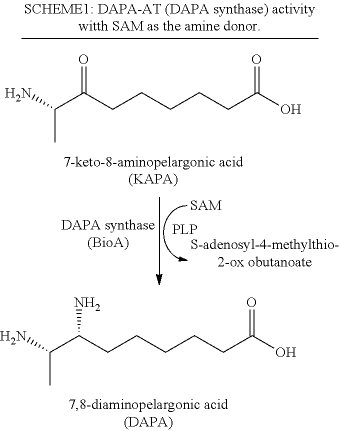

The present invention is based on the development of a new assay in which an alternative amine donor is utilised.

Thus, according to the present invention there is provided an in vitro method for determining whether or not a candidate compound is an inhibitor of diaminopelargonic acid aminotransferase (DAPA-AT) said method comprising (i) assaying the candidate compound in an assay mixture comprising (a) DAPA-AT, (b) an amine donor, (c) pyridoxal 5′-phosphate (PLP) and (d) 7-keto-8-aminopelargoic acid (KAPA), and (ii) detecting inhibition of the DAPA-AT by the candidate compound; characterised in that the amine donor comprises an aliphatic diamine or aliphatic polyamine or a carbamate derivative thereof.

By aliphatic diamine it is meant a low molecular weight aliphatic compound containing two amino (NH₂) groups, for example cadaverine or putrescine. By aliphatic polyamine it is meant a low molecular weight aliphatic compound containing more than two amino (NH₂) groups, for example spermidine or spermine. Thus, in a preferred embodiment of the present invention the amine donor is a compound of Formula (I):

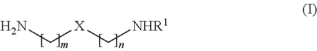

wherein X is NH or CH₂;
R¹ is hydrogen or —[CH₂]pNH₂;
m and n are independently selected from 0, 1, 2, 3 or 4; and
p=1, 2, 3 or 4,
or a carbamate derivative thereof wherein R¹ is —C(O)OH.

In another embodiment of the present invention, the amine donor is selected from the group consisting of cadaverine (X=CH₂, R¹=H, m=2, n=2), putrescine (X=CH₂, R¹=H, m=2, n=1), spermidine (X=NH, R¹=H, m=3, n=4) and spermine (X=NH, R¹=—[CH₂]ₚNH₂, m=3, n=4, p=3). In a particularly preferred embodiment of the present invention, the amine donor is spermidine.

It has been found that the use of aliphatic diamine or polyamine donors (as opposed to SAM), especially spermidine, can provide a significantly improved assay, especially with regard to assays utilising plant BIO1 or BIO3-BIO1 enzymes (See FIG. 1). Without being bound to any particular theory, it is believed that when spermidine is utilised in the assay, it is converted to 4-(3-aminopropylamino)butanal (or 3-(4-aminobutylamino)propanal) (Scheme 2).

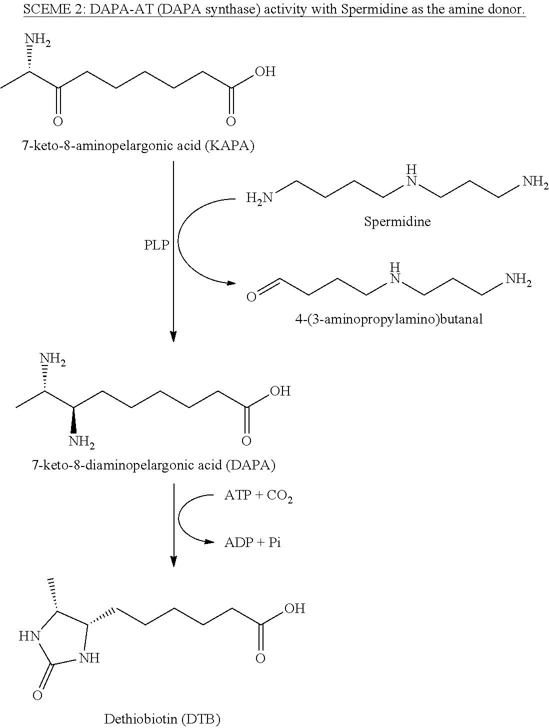

The provision of a new functional assay is thus particularly useful for methods of identifying inhibitors of DAPA-AT, especially plant BIO1 or BIO3-BIO1, which could help in the discovery and development of herbicidal compounds.

The candidate compound can be any chemical compound that is a putative inhibitor of DAPA-AT. Examples of known DAPA-AT inhibitors that can be used to develop the method include: a herbicidal cinnoline compound such as ethyl 5-acetyl-4-oxo-1-(2,2,3,3-tetrafluoro-1,4-benzodioxin-6-yl)cinnoline-3-carboxylate (Compound A); a herbicidal compound such as [5-carboxy-1-(1-hydroxyethyl)pentyl]ammonium; chloride (compound B); a herbicidal pyridone compound such as 2-(3,4-dichlorophenyl)-5-(2,4-difluorophenyl)-1-ethyl-6-methyl-4-oxo-pyridine-3-carboxylic acid (Compound C) or 5-[2-chloro-5-(trifluoromethoxy)phenyl]-2-(3,4-dichlorophenyl)-1-ethyl-6-methyl-4-oxo-pyridine-3-carboxylic acid (Compound D); a herbicidal compound such as [(1R)-2-(6-carboxypyridin-1-ium-2-yl)oxy-1-methyl-ethyl]ammonium; dichloride (Compound E); a herbicidal pyrrolidine-2-one such as 2-[1-[(2,3-difluorophenyl)methyl]-5-oxo-pyrrolidin-2-yl]-N-(2-methyl-1,2,4-triazol-3-yl)acetamide (Compound F), 2-[1-[(2,3-difluorophenyl)methyl]-5-oxo-pyrrolidin-2-yl]acetic acid (Compound G), 2-[5-oxo-1-[(2,3,5-trifluorophenyl)methyl]pyrrolidin-2-yl]acetic acid (Compound H), 2-(4-fluorophenoxy)ethyl 2-[1-[(2,3-difluorophenyl)methyl]-5-oxo-pyrrolidin-2-yl]acetate (Compound I), 2-methoxyethyl 2-[1-[(2,3-difluorophenyl)methyl]-5-oxo-pyrrolidin-2-yl]acetate SUBSTITUTE SHEET (RULE 26) (Compound J), 2-[5-oxo-1-[(2,3,4-trifluorophenyl)methyl]pyrrolidin-2-yl]acetic acid (Compound K); a N-benzyl azole herbicidal compound such as 2-(3,4-dichlorophenyl)-1-ethyl-4-oxo-6-[[3-(trifluoromethyl)pyrazol-1-yl]methyl]pyridine-3-carboxylic acid (Compound L); and a herbicidal quinolone compound such as 2-(3,4-dichlorophenyl)-1-ethyl-6-fluoro-4-oxo-quinoline-3-carboxylic acid (Compound M). Results obtained using these compounds to develop the method of the present invention are shown in Table 1; and typical IC50 plots shown in FIG. 2.

The assay mixture comprises (a) DAPA-AT, (b) an amine donor, (c) pyridoxal 5′-phosphate (PLP) and (d) 7-keto-8-aminopelargoic acid (KAPA). The term diaminopelargonic acid aminotransferase (DAPA-AT) as represented by component (a) refers to an enzyme that catalyses the conversion of 7-keto-8-aminopelargonic acid (KAPA) to 7,8 diaminopelargonic acid (DAPA). Examples of DAPA-AT could include, for example, bacterial BioA enzymes or functional derivatives or fragments thereof. More preferably, the DAPA-AT is a plant BIO1 enzyme, which can be present in the form of a bifunctional BIO3-BIO1 enzyme or a functional derivative or fragment of the BIO1 or BIO3-BIO1 enzyme. BIO3-BIO1 refers to an enzyme that is capable of catalysing the conversion of KAPA into dethiobiotin. The exact nature of the DAPA-AT is not germane to the invention, and many suitable DAPA-AT enzyme sequences are reported in the literature. Thus, the methods of the present invention can utilise various plant BIO3-BIO1 enzymes including, for example, the mature BIO3-BIO1 enzyme from a monocotyledon plant species (e.g Setaria italica—for results see FIG. 3) and the mature B103-BIO1 enzyme from a dicotyledon species (e.g Arabidopsis thaliana—for results see FIG. 1).

Thus, in a preferred embodiment of the present invention the DAPA-AT is a plant BIO1 or a functional derivative of fragment thereof. In a more preferred embodiment, the DAPA-AT is provided within a bifunctional BIO3-BIO1 enzyme or a functional derivative or fragment thereof. In a preferred embodiment, the DAPA-AT is a BIO3-BIO1 enzyme selected from the group consisting of SEQ ID NOs 1 to 37 or a functional derivative or fragment thereof. In one embodiment of the present invention, the DAPA-AT is the mature Arabidopsis BIO3-BIO1 (SEQ ID NO. 21) or Setaria BIO3-BIO1 (SEQ ID NO. 25) or a functional derivative or fragment thereof. In an embodiment of the present invention, the DAPA-AT is a plant BIO3-BIO1 enzyme which is not from Arabidopsis.

By functional derivative it is meant a variant in which some of the amino acids in the wildtype DAPA-AT have been substituted and/or deleted but wherein the DAPA-AT activity is retained. Typically, such functional derivatives will retain 85%, more preferably 90%, even more preferably 95%, even more preferably 99% sequence identity at the amino acid level.

By fragment it is meant a truncated version of the DAPA-AT enzyme wherein the DAPA-AT activity is retained. For example, wildtype BIO3-BIO1 enzymes include a N-terminal mitochondrial targeting peptide (22 amino acids with regard to the Arabidopsis BIO3-BIO1 of SEQ ID NO. 1, for example) and it should be understood that this can be removed such that the truncated, mature DAPA-AT is used in the methods of the present invention. Examples of B103-BIO1 enzymes with the putative N-terminal mitochondrial targeting peptides removed are provided as SEQ ID NO. 21 to 37.

It is envisaged that DAPA-AT can be provided for use in the assay in many art-recognised ways. For example, it could be provided in the form of a crude extract, but more preferably the DAPA-AT will be produced by recombinant means and used in the assay in a suitably purified form.

The amine donor can be obtained from any suitable source. For example, it may be obtained from a suitable biological source and contained, for example, in a crude extract, or a clarified biological lysate. Alternatively, and preferably, the amine donor may be chemically synthesised, and thus provided in the assay mixture in a suitably pure form. It should be understood that the component (b) may contain more than one amine donor.

Since DAPA-AT is a PLP-dependent enzyme then is necessary to further include (c) PLP in the assay mixture. PLP may already be present in the bound to DAPA-AT and may thus be provided in the assay mixture in this manner. Alternatively, exogenous PLP can be provided in the assay mixture, which is readily available form commercial sources.

The substrate 7-keto-8-aminopelargoic acid (KAPA) (d) is likewise readily available from commercial sources.

The skilled person will understand that further assay components will be included in the assay mixture. These could include, for example, buffers such as 3-[4-(2-Hydroxyethyl)piperazin-1-yl]propane-1-sulfonic acid (EPPS), 4-(2-hydroxyethyl)-1-piperazineethanesulfonic acid (HEPES), N-[1,3-Dihydroxy-2-(hydroxymethyl)propan-2-yl]glycine (Tricine) or 2-Amino-2-(hydroxymethyl)propane-1,3-diol (Tris), salts such as sodium chloride, potassium chloride or magnesium chloride, detergents such as Polyoxyethylene (20) sorbitan monolaurate (Polysorbate 20), 2-[4-(2,4,4-trimethylpentan-2-yl)phenoxy]ethanol (Triton X-100) or 3-{Dimethyl[3-(3α,7α,12α-trihydroxy-5β-cholan-24-amido)propyl]azaniumyl}propane-1-sulfonate (CHAPS), reducing agents such as (2S,3S)-1,4-Bis(sulfanyl)butane-2,3-diol (dithiothreitol), chelating agents such as N,N′-(Ethane-1,2-diyl)bis[N-(carboxymethyl)glycine](EDTA) or other proteins such as Bovine serum albumin (BSA). The exact nature of these routine components is not germane to the invention.

Conveniently, the method of the present invention will be carried out in at least two distinct stages. In the first stage, the candidate compound is introduced into an enzyme mixture comprising the (a) DAPA-AT. The enzyme mixture is then optionally incubated for a period of time to allow the candidate compound to bind to the DAPA-AT. In the second stage, the assay mixture is provided by the addition of a substrate mixture which comprises the amine donor (b) and KAPA (d) to the enzyme mixture. The PLP (c) will typically be included in the enzyme mixture but can be additionally/alternatively provided in the substrate mixture. The assay will typically be conducted at room temperature for 60 minutes.

The “detecting inhibition” part of the method can be conducted using several art-recognised methods that would be readily apparent to the skilled person. For example, options include direct detection in these functional assays of DAPA formation, or KAPA loss or by coupling the assay to dethiobiotin synthetase (DTBS) and detecting the formation of the product, DTB. In the situation that BIO3-BIO1 is utilised in the assay, it is possible to further include (e) ATP and (f) CO₂ in the assay mixture and detect the formation of DAPA and/or DTB. These components can be monitored using, for example, suitable LC-MS procedures. Other assays including radiolabelled and spectrophotometric assays are also envisaged. However, in a preferred embodiment of the present invention the formation of DAPA is monitored in the assay. In a more preferred embodiment, the DAPA is conveniently converted to a fluorescent derivative, for example DAPA:o-phthalaldehyde.

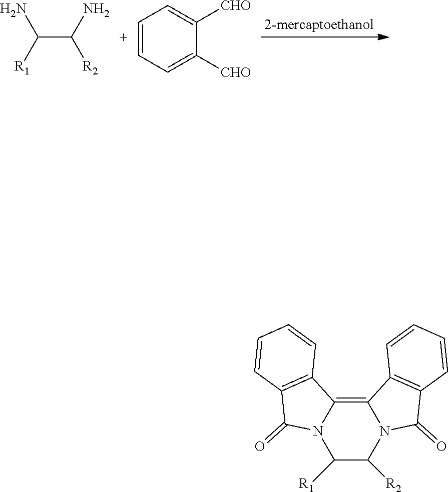

The use of a fluorescent derivative in the method of the present invention is particularly useful, as it lends itself to high-throughput assays using, for example, using multi-well plates. Thus, in a particular embodiment of the invention the assay is terminated using a termination mixture comprising o-phthalaldehyde and 2-mercaptoethanol.

Inhibition can be determined by comparing the results obtained from the assay with results obtained from an otherwise identical assay conducted in the absence of the candidate compound. % inhibition can then be calculated using the following calculation, % inhibition=100×((((MAX−MIN)−(X−MIN))/(MAX−MIN))), where MIN and MAX are the average of the minimum reaction and maximum reaction controls and X is the sample of interest, in order to characterise the candidate compound as an inhibitor or not of DAPA-AT.

The present invention further provided the use of an amine donor which comprises a compound of Formula (I):

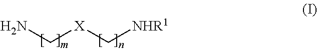

wherein X is NH or CH₂;
R¹ is hydrogen or —[CH₂]pNH₂;
m and n are independently selected from 0, 1, 2, 3 or 4; and
p=1, 2, 3 or 4;
or a carbamate derivative thereof wherein R¹ is —C(O)OH;
in an in vitro diaminopelargonic acid aminotransferase (DAPA-AT) assay.

### EXAMPLES

The following non-limiting examples provide specific methods which are representative of the present invention.

### Example 1—Expression of an Arabidopsis thaliana BIO3-BIO1 (BioDA) in E. coli

A pET-24 plasmid containing a DNA sequence encoding BIO3-BIO1 derived from Arabidopsis thaliana, codon-optimised for expression in E. coli, with the predicted N-terminal mitochondrial transit peptide removed and with an N-terminal 6×His-tag and thrombin cleavage site and under control of a T7 promoter (SEQ ID NO:38), was synthesised by Twist Bioscience. The plasmid was expressed in E. coli BL21 (DE3) bioA with 50 μg/mL kanamycin and 25 μg/mL chloramphenicol selection. Overnight cultures grown at 37° C. were used to inoculate 8×500 mL AIM in shake flasks at a ratio of 1:100. Cultures were grown at 37° C., 200 rpm for 3.5 hours and then overnight at 20° C. Cells were harvested by centrifugation for 30 minutes at 10 000 g. Cells were stored at −80° C. until extraction. 30 g of cell pellet from was thawed in 120 mL of IMAC buffer A (20 mM HEPES pH7.5, 25 mM imidazole, 500 mM sodium chloride, 0.15 mM pyridoxal phosphate, 0.5 mM TCEP, 1×complete Protease Inhibitor Tablet (Roche)). The resuspension was passed through a cell disrupter at 18 000 PSI and centrifuged at 50 000 g for 25 minutes at 4° C. The His-tagged BIO3-BIO1 protein was purified from the extract supernatant on an ÄKTA avant chromatography system (Cytiva) using a HisTrap FF column and eluted in IMAC buffer B ((20 mM HEPES pH7.5, 500 mM imidazole, 500 mM sodium chloride, 0.15 mM pyridoxal phosphate, 0.5 mM TCEP) followed by SEC using a HiLoad 26/60 Superdex200 column in SEC buffer (25 mM HEPES pH7.5, 150 mM sodium chloride, 0.15 mM pyridoxal phosphate, 0.5 mM TCEP). Protein concentration was measured and calculated using the absorbance at 280 nm. Extracts were diluted 1 in 4 and 10 uL was analysed by SDS PAGE.

### Example 2—DAPA-AT Assay

To measure DAPA-AT enzyme activity in the presence of herbicide (candidate compound), 0.5 μL of the herbicide was incubated with 20 μL of enzyme mix (100 mM EPPS pH 8.6, 0.1 mM pyridoxal phosphate, 0.1 mg/mL BSA, 25 nM BIO3-BIO1 enzyme) for 10 minutes at 20° C. in a well of a 384-well black plate, followed by the addition of 20 μL of substrate mix (final concentration of 100 mM EPPS pH 8.6, 0.1 mM pyridoxal phosphate, 0.1 mg/mL BSA, 10 μM spermidine, 1 μM KAPA, 100 mM NaHCO₃) and further incubation for 60 minutes at 20° C. The reaction was terminated by the addition of 50 μL of derivatisation mix consisting of 185 mM borate NaOH pH 9.4, 3 mM o-phthalaldehyde, 6 mM β-mercaptoethanol, 22.5% (v/v) ethanol, 3% (v/v) methanol. The fluorescent product produced by the reaction of DAPA with o-phthalaldehyde and β-mercaptoethanol was measured at 470 nm after excitation at 410 nm. The percentage inhibition was calculated using the equation, % inhibition=100×((((MAX−MIN)−(X−MIN))/(MAX−MIN))), where MIN and MAX are the average of the minimum reaction and maximum reaction controls and X is the test sample. Compounds are typically added directly to the assay plate in a serial 3× dilution from a top final concentration of 100 μM over 10 different concentrations. Example results are shown in Table 1 below. The percent inhibition values are fitted to a dose-response using the equation Y=Bottom+(Top−Bottom)/(1+((X^{Hill Slope})/(IC₅₀^{Hill Slope}))) to calculate the compound concentration that gives 50% inhibition (IC₅₀).

TABLE 1
% Inhibition of mature Arabidopsis BIO3-BIO1 (SEQ ID NO: 21) with various inhibitor compounds.

| Inhibitor | Compound A | | | Compound B | | | Compound C | | | Compound D | | |
|---|---|---|---|---|---|---|---|---|---|---|---|---|
| Concentration | 1 μM | 5 μM | 25 μM | 20 μM | 100 μM | 200 μM | 0.2 μM | 1 μM | 5 μM | 0.2 μM | 1 μM | 5 μM |
| % Inhibition | 59 | 84 | 96 | 84 | 95 | 96 | 29 | 64 | 85 | 51 | 84 | 95 |

| Inhibitor | Compound E | | | Compound L | | | Compound M | | |
|---|---|---|---|---|---|---|---|---|---|
| Concentration | 20 μM | 100 μM | 200 μM | 0.2 μM | 1 μM | 5 μM | 0.2 μM | 1 μM | 5 μM |
| % Inhibition | 80 | 94 | 96 | 6 | 44 | 83 | 23 | 58 | 82 |

### Example 3—Measuring DAPA-AT and Dethiobiotin Synthetase Enzyme Activity in the Presence of Different Amine Donors

To measure DAPA-AT and dethiobiotin synthetase enzyme activity in the presence of different amine donors 10 μL candidate amine donor was placed in a well of a 96-well plate (final concentration 3.3 mM). 10 μL of substrate mix (final concentration 100 mM EPPS pH 8.6, 0.1 mM pyridoxal phosphate, 1 mM ATP, 5 mM MgCl₂, 20 μM KAPA, 100 mM NaHCO₃) was added to each well. The assay was initiated by the addition of 10 μL BIO3-BIO1 enzyme (final concentration 1 μM) and incubated at 20° C. for up to 90 minutes. The reaction was stopped by addition of 100 μL 1:1 acetonitrile/methanol and the dethiobiotin (DTB) was quantitated by LC-MS using an Orbitrap mass spectrometer in positive ionisation and full scan mode, using a heated electropspray ionisation source. Analytes were separated by reverse phase using a UPLC 2.1 mm by 50 mm C18 column. 5 μL of the sample was injected onto the column equilibrated with 95% solution A (0.2% formic acid) and 5% solution B (acetonitrile) at a flow rate of 0.7 ml/min. The DTB was eluted with a 0.7 ml 5 to 95% B gradient and compared to a commercial standard of DTB. The results obtained are summarised in FIG. 1.

### LIST OF FIGURES

FIG. 1 Arabidopsis BIO3-BIO1 activity comparing various amine donors.

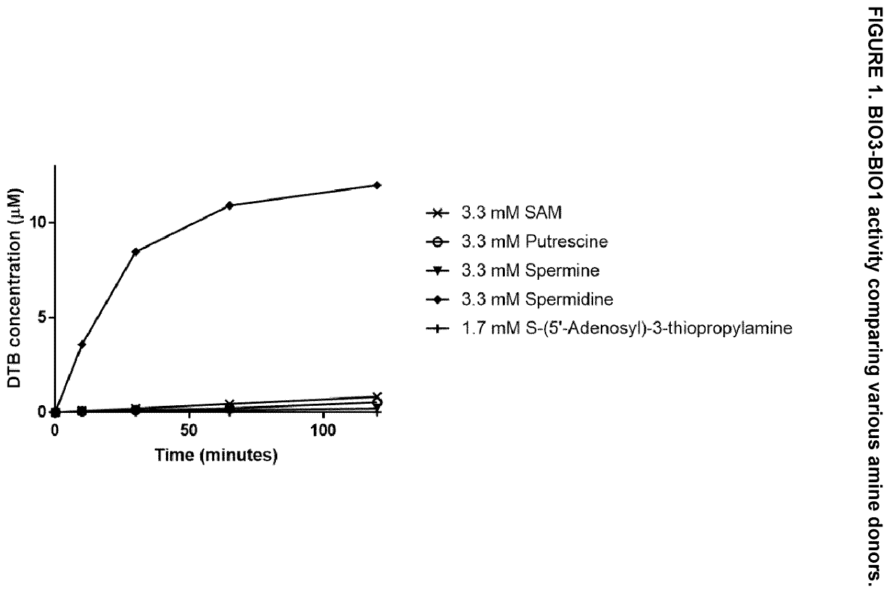

FIG. 2 Typical IC50 Plot.

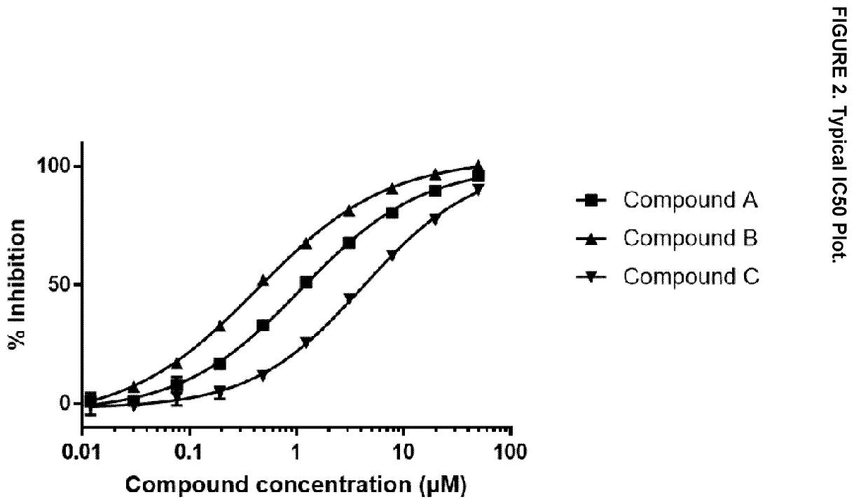

FIG. 3 Setaria BIO3-BIO1 enzyme activity at different amine donor (spermidine) concentrations.

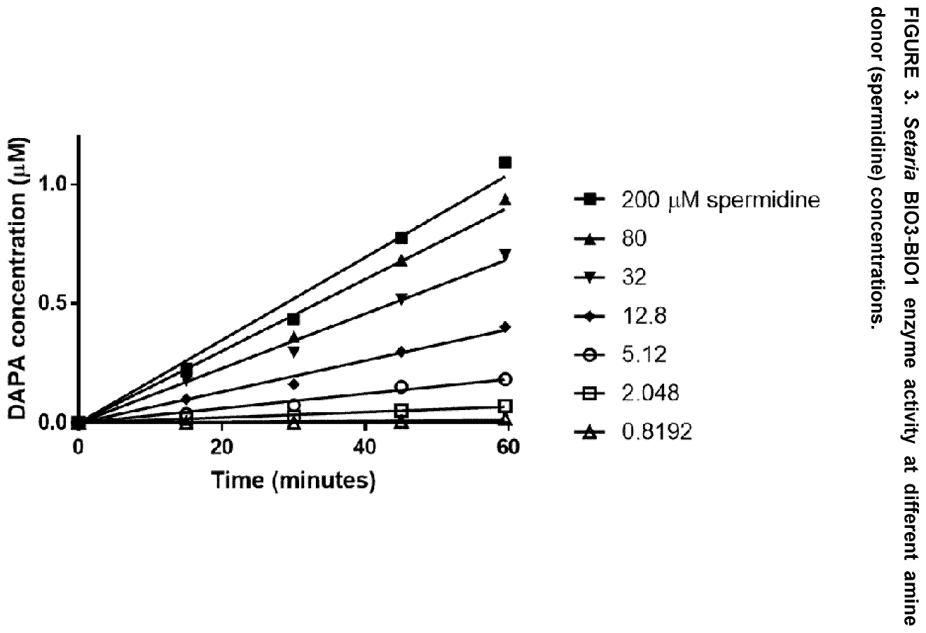

1. An in vitro method for determining whether or not a candidate compound is an inhibitor of diaminopelargonic acid aminotransferase (DAPA-AT) said method comprising (i) assaying the candidate compound in an assay mixture comprising (a) the DAPA-AT, (b) an amine donor, (c) pyridoxal 5′-phosphate (PLP) and (d) 7-keto-8-aminopelargoic acid (KAPA), and (ii) detecting inhibition of the DAPA-AT by the candidate compound; characterised in that the amine donor comprises an aliphatic diamine or aliphatic polyamine or a carbamate derivative thereof.

2. A method according to claim 1, wherein the DAPA-AT is a plant BIO1 or a functional derivative or fragment thereof.

3. A method according to claim 1, wherein the DAPA-AT is provided as a bifunctional BIO3-BIO1 enzyme or a functional derivative or fragment thereof.

4. A method according to claim 1, wherein the DAPA-AT is the Arabidopsis BIO3-BIO1 or a functional derivative or fragment thereof.

5. A method according to claim 3, wherein the DAPA-AT is SEQ ID NO:21 or a functional derivative or fragment thereof.

6. A method according to claim 1, wherein the DAPA-AT is recombinantly produced.

7. A method according to claim 1, wherein the amine donor is a compound of Formula (I): 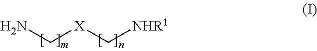
wherein X is NH or CH₂;
R¹ is hydrogen or —[CH₂]pNH₂;
m and n are independently selected from 0, 1, 2, 3 or 4; and
p=1, 2, 3 or 4,
or a carbamate derivative thereof wherein R¹ is —C(O)OH.

8. A method according to claim 1, wherein the amine donor is selected from the group consisting of cadaverine, putrescine, spermidine and spermine.

9. A method according to claim 8, wherein the amine donor is spermidine.

10. A method according to claim 1, wherein the detecting inhibition comprises monitoring the formation of diaminopelargonic acid (DAPA).

11. A method according to claim 1, wherein the method utilises a assay mixture wherein the candidate compound is first incubated with the DAPA-AT (a) and PLP (c) in an enzyme mixture and second combining the enzyme mixture with a substrate mixture comprising the amine donor (b) and the 7-keto-8-aminopelargoic acid (KAPA) (d).

12. A method according to claim 1, wherein the formation of DAPA is monitored by converting the DAPA to a fluorescent derivative.

13. A method according to claim 12, wherein the fluorescent derivative is DAPA:o-phthalaldehyde.

14. A method according to claim 3, wherein the assay mixture further comprises (e) ATP and (f) CO₂ and the detecting inhibition comprises monitoring the formation of DAPA and/or dethiobiotin (DTB).

15. Use of an amine donor which comprises a compound of Formula (I): 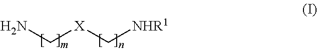
wherein X is NH or CH₂;
R¹ is selected from the group consisting of hydrogen, —C(O)OH and —[CH₂]ₚNH₂;
m and n are independently selected from 0, 1, 2, 3 or 4; and
p=1, 2, 3 or 4;
or a carbamate derivative thereof wherein R¹ is —C(O)OH;
in an in vitro diaminopelargonic acid aminotransferase (DAPA-AT) assay.


SEQUENCE LISTING (38 sequences)

SEQ ID NO: 1 - protein, 833 aa, Arabidopsis thaliana
MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKLDSLSLRRQIPISISNSVLHSSLPAAKSLGLNVEVSESGMCSLNFRDEKTVTGAPELLCKTLYAWEAAISPHLAAERENATVEDSVVLQMIEKCLKEEMECGVKSEKSDLLCLVETAGGVASPGPSGTLQCDLYRPFRLPGILVGDGRLGGISGTIAAYESLKLRGYDIAAVVFEDHGLVNEVPLTSYLRNKVPVLVLPPVPKDPSDDLIEWFVESDGVFKALKETMVLANLERLERLNGMAKLAGEVFWWPFTQHKLVHQETVTVIDSRCGENFSIYKASDNSSLSQQFDACASWWTQGPDPTFQAELAREMGYTAARFGHVMFPENVYEPALKCAELLLDGVGKGWASRVYFSDNGSTAIEIALKMAFRKFCVDHNFCEATEEEKHIVVKVIALRGSYHGDTLGAMEAQAPSPYTGFLQQPWYTGRGLFLDPPTVFLSNGSWNISLPESFSEIAPEYGTFTSRDEIFDKSRDASTLARIYSAYLSKHLQEHSGVRQSAHVGALIIEPVIHGAGGMHMVDPLFQRVLVNECRNRKIPVIFDEVFTGFWRLGVETTTELLGCKPDIACFAKLLTGGMVPLAVTLATDAVFDSFSGDSKLKALLHGHSYSAHAMGCATAAKAIQWFKDPETNHNITSQGKTLRELWDEELVQQISSHSAVQRVVVIGTLFALELKADASNSGYASLYAKSLLIMLREDGIFTRPLGNVIYLMCGPCTSPEICRRLLTKLYKRLGEFNRT

SEQ ID NO: 2 - protein, 819 aa, Zea mays
MVRRLFLHHARRRLHSGTASSIPLSTPIFAIFGANTGVGKTLVSAGLATALLSSPSPAVSSVAYLKPLQTGYPVDSDASFVFSRTPALLRAFPPCRATRLIASCRTFFQSPAVEAKAESLHESQEKVVTYGAGAAEQTKVLACCTAYAWREPVSPHLAAEREGMAAGDDEVKGCVEQWLLEEGIGEGGEVWKILETAGGVASPSASGTLQCDLYRPFRLPAILVGDGRLGGISSTLSAYETLLLRGYDVSAVILEDRELSNDKFLLSYLRNRVHVLILPQIPEDPLDDLTDWFSESSSIFIFLKDELQSFHSRRIERLNSMQRKSKALLWWPFTQHNLVPQDSVTVIDSRYGENFSVYKIKDKMLIPQFDACASWWTQGPDSNLQIELARDMGYAAARYGHVMFPENVHEPALHSAEVLLGGVGKGWASRVYYSDNGSTAIEIALKMAFRKFSLDHGIMASSENSTRNERNIQLKVLALNGSYHGDTLGAMEAQAPSAYTSFLQQPWYSGRGLFLDPPTVYIKSEVYNISLPQSMQHDPQTYGFFSQAEVFCKTRDKTFAADLYSSYIKQKLSEFSLSSSSEHLAALIIEPVIQGAGGMLMIDPLFQRVLVSECRSRKIPVIFDEVFTGFWRLGVESASELLGCLPDIACYAKLMTGGIIPLAATLATEEVFESFESDSKLTALLHGHSYTAHAMGCTAALKAMQWYRGPSTNSNLDTDHMKLKELWDGTLVKQLSSLPNVKRVVSLGTLCAIELKAEGADAGYASLYASSLVQQLRKEDDIYIRPLGNVIYLMCGPCTPRDSCTRQLLKVHRRLCALN

SEQ ID NO: 3 - protein, 859 aa, Nannochloropsis gaditana
MCSLTIQVFGANTDVGKTVISAGLLRAASNRPLRRRTTYIKPLQTGKIHDGSFVQRYTSADVSCHTLYSWETPLSPHVAATKEGKTISSEELLGMLRTCLQQAQESHTVGGDGGGVRQREGSNVGDLVVVETAGGVLSPGPSPLQTQADLYRPLRLPVLLVGDGRLGGISSTLAAYESLYCRGYEVHAIAVLEVPHYIENAAAVSEYITGTLGHAETPVLSLPHLPEVSSVSFSTGDVDKDVLLGGLDQWYHQHEHVFHHLLDTLIDRERVRIQRLQKMTTQAAQVFWYPFTQHSGLSGTDLHVVDSAYGDNYTLISKSSEDGDAKERVPCSRTLIDACASWWTQGLGHGRVDVALAVGAAAGRFGHVIFPGNVHEPALRLAELMLAGPGQGWASRVFFSDNGSTAMEIAVKMALKKNSVDLKYEKGCSKEEFRVEQPNDKISATPLTVVTQRDCYHGDTLGCMDAAAPTIFNTGQHPWYQPRTLSLTVPTFGYVSGHLQVEIPPEITRAAGQSALQLPDSGQVLKFPGGLATLLDVHAREGSDAAVIYRKAIRAQFIAFERGEEGILSSNTVIPLRGEAGQGHRCRKLGAVLLEPVLMGAAGMVMVDPLFQRILTLEGRAYGLPVVFDEIASGLYRLGCASAAELLGVAPDIACYGKTLTGGYLPMALTLTTEAVFEAFHGTNKSDALLHGHSYTANPLGCAAAVHALKAYAVIHETNIRTSQECCQRKEDFFPNKNKCSSHHNICRYNEDQVAALSNLSGVRRAVALGTVLAVELTEVESSNRKGYSSTASTHIVNDLREAGIYARPLGNVVYLIASQVSSGGTFECLVDTLDRVLRRRGDTLGKFTHDMTTSGVVI

SEQ ID NO: 4 - protein, 811 aa, Taxus chinensis
MASLALTRRRFNPVSRLSLHKCRSTFSRSGQAGEDRTPKQKLHIPSGFEVDLSHPTYVIWGSNTGVGKTLVSTGLAASLLNASQNLLYVKPVQTGFPQDSDSRYVSHKVKDILKQRKPSNNVHISHQVLKASPRAWELLPGSKGFGEVASYERAIQGMRNVCDYEGQTVQGLYKGLADLKLVCKTIYGWYEAVSPHLAAAGEKCSVDDCEVVENLQNSLREFNVDENNTNRWSIIETAGGVASPGPSGTLQCDLYRPFRLPSILVGDGRLGGISGTISAYETLCIRGYDICAVILADTGLSNEKCLSTYLHDRVPVFVLPKIPDDTNNTLLEWFSESYKVFSEIKDTLQTSYFKRIYRLHEMPMKARQILWWPFTQHSLVTEQSISLIDSRFGENFGVYKGKNHKDVKESIVQQFDACASWWTQGPSAILQTELAREVGYTTGRFGHVMYPENVYEPALHCAELLLEGVGKGWATRVFLSDNGSTAIEISLKMAFRKFAFDHGFLPEHFNEDLRKICPQFKVEIGTISYTTCAFFIEMTMEEFSEESYTGRGLFLDPPTVFLHNEEWQLHLPDALMYSDLKYPGLVIHASGGMHMIDPLFQRDLVKECRKRRIPVIFDEVFTGFWRLGAESAAEILGCAPDIACYAKLLTGGIVPLAATLATESIFEAFEGKSKLFALLHGHSYSGHAIGCAAAIKAIQWFKDAEMNLNICTDGSRLQELWDSKLVAKISALSSVKRVIALGTICALELQADESNTGYGSLLASSLVQKLRDDGVYMRPLGNVIYLMCGPATPPAMCSTLLQKLHKRLDEC

SEQ ID NO: 5 - protein, 928 aa, Physcomitrium patens
MHLLLLLPLRRRCTNPIAPRIAHQSRFLVSTAGACSPLPRHLLSGIWGRCLSSHPSPIEDADEARCVAENVADALDVSYPTYMVWGSNTGVGKTLVSAGLCSAILRLSKCQGNSGAKDLLYLKPVQTGFPSDSDARYVYQEVSRVGRLHPEAISEGLFLCNHTLQVSPAVQISARAGASVPVQPQSAWNAGGLSSSLNGTGMKDFCSYESQLLQGVKSRNIPADGDVYGRAQLRNTLSRFTCKTMWAWYEPISPHLAAAREGSTVPDTMVVEAIQRSLQALSSSRKTSGHREDAGDCAEKWAILETAGGVASPGPSGSLQCDMYRSLRFSGLLVGDGRLGGISTTIAAYESLRSRGYDIPAIVIVDSGLSNEEPVLQYLRHKIPVVVLPPLPEDPNKRLEEWFRLAEGTFLELMNVLENSHSKRLRRLEEMPKMAGEILWWPFTQHDLVAQDSITLIDSRSGENFSVYKSGTPSTGKTIATQFDACASWWTQGPDSILQPEIAREVAYTAGRYGHVMFPENVHEPALRCAEILLAGAGRGWAERVFFSDNGSTAIEVAIKMAFRKYIVDHGLSDPSTIERRGGQLKVVALKGSYHGDTLGAMEAQAPSAYTGFKQQPWHVGRGFFLEPPTVFQRQGRWHLQLPRVFHDSDSSDSASCWKSRDEIFDICRDSTPLANLYIRSINQQLAAAVREENNVPAALIIEPVMHGAGGMDMIDPLYQRMLVRECRRRGIPTIFDEVLSGCWRFGVESAVDLLGCSPDIACYSKLLSGGLVPLAATLATQAVFEAFRGISKLDALLHGHSYTAHAVGCASAVVSLQYFSDPSKNSNLLPGGRRLRELWKPELVSLISSHQSVERVVSVGTIFALELRANAAESGYASMLSKELVENLRQEGIYTRPLGNVVYLMCGPLTQPKTCTTMLEKLLALLG

SEQ ID NO: 6 - protein, 903 aa, Adiantum nelumboides
MAPPLSHLYPHLRIHQIFGANTDVGKTIFTTALALASAALPIGSKNGKIERDGQGSLNFQENGEAVHYIKPISTGPLRDSDSLHIARYLPSVNAHTLFQFNDPVSPHVAAARYKAEDKNKQDLDVSDEAVIHALSNKISTFTQAAIRPGAAYIETAGGVHSPAPSGSSISQLLRPLRMPTILIGDSNLGGISTTKSAYDSLLMAGYDIEALLLFSDAGRGWGNAEYLTKWGEEIGLPVWALTGPSSSTVWGLPPAREGSDEEDARRMQEYYAGLVAGRGDAAENDESSLGAAPIIEHLRRRHEKRLENIGSMAKRTRDTCWWPFTQHMLAKSDEDVNVIDSAYGDFFTVLNQSSENASKHGNTSLLRPLLDGSASWWTQCLGHADPGLTRAAARAAGRYGHVLFPMCANEPSLQLSETLVGKNSSDVIGTSPGEGWASRVFFSDDGSTGMEVALKMAISSASSRYAPKAESQLAKSKVQQGRSAGNQSGRPPREWMVLGLKGSYHGDTIGAMDACEGNIFNEKVEWYRGRGSWFEPPTVSIKDGQAMVDLPLQDADWQHLSGKVESKKNFNNVSDIYNVSDRMDNDELKNIYSKLIYGWLDHLTRMEGNRYGALILEPLIMGAGGMICVDPLFQRCFVDVIRANPDLFSQVDPPLRGTSHIRESDVPGSGDWKGLPIIFDEVFTGLYRLGYSTPAQAIGVSPDISVLAKILTGGMVPMSVTLASDSIFSTFSKSDQKIDALLHGHSYTAHPIGCEIANEALRRIETMQVENFWGDAKADWESRTSSLQRDNSMKNVPSPWSLWSREAVEAFSRSANVESSMALGTVMILHLSDSAGEKGYSSSAAVDILQHLRTTQDTGFNKHVSFNIHARPLGNVVYMMCSLNTPVNVLRETEAALKHVLKV

SEQ ID NO: 7 - protein, 825 aa, Setaria italica
MLPRLLLRSRHRRRYSSAAATVPLSTPTFAVFGANTGVGKTLVSAGLVAALLNSRSPSVSAVSYLKPLQTGYPADSDARFVFARAPALLRASSSPRATRLVASCRTLFPSPAVGPEAEPLHQSQEKVVTYGGDGAEEETKVLACRTVYAWREPVSPHLAAEREGMAAGDDEVRGCVDQWLMEEGVGEGGEVWKVLETAGGVASPSASGTLQCDLYRPFRLPAILVGDGRLGGISSTLSAYETLLLRGYDVSTVILEDRDLTNDTFLLSYLRNRVHVLALPQIPEDPSDDLTDWFSESSSVFNLLKDSLQSFHSKRVERLNGMQKKSKDLLWWPFTQHNLVPQDSVTVIDSRCGENFSAYKIKDNTMMLVPQFDACASWWTQGPDSDLQIELARDMGYAAARYGHVMFPENVHEPALRAAELLLGGVGKGWASRVYYSDNGSTAVEIALKMAFRKFSLDHGILADSEKSIRNERNIQLKVLALNGSYHGDTLGAMEAQAPSAYTSFIQQPWYSGRGLFLDPPTVCIKSETCNVSLPQSMQHYPRTSGDTCFPSLAEVFCKTRDTSSAADLYLSYIKQQLSEFSQSSNSDHLAALIIEPVIQGAGGMLMIDPLFQRVLVRECRSRKIPVIFDEVFTGFWRLGVESASELLGCLPDIACYAKLMTGGIVPLAATLATEEVFESFKSDSKLTALLHGHSYTAHAMGCMAALKAIQWYRDPSTNSNLDLDHMKLKELWDGALVKQLSSLPNVKRVVSLGTLCAIELKAEGSDAGYASLYASSLVQQLRREDDIYIRPLGNVIYLMCGPCTPRDSCSRQLHKVLRRLCDLN

SEQ ID NO: 8 - protein, 870 aa, Phoenix dactylifera
MPSCPLLPLLPRRRPPPSPLLLLLLSRSLHSSPPELDLSHPTLVVWGSNTGVGKTIVSAGLAASVLSPSSPNTATFLYLKPVQTGFPRDSDSRFVYRKVSDLFRHRLGGFSAQLLASNHALKASVPAAREVLGARTERPGEEGEEASTGLCCYEETRVGTGESTTGDFKLVCKTLYGWKEAISPHLAVEREGMAVEDSSLREVLGRLLNSSMGGGGDRRKENVWRVIETAGGVASPGPSGSIQCDLYRPFRLPAVLVGDGHLGGISTTLSAYESLTLRGYDVIAIILADHGLSNEIPLLSNLRHRVPVLVLPPVPEDPLNDLVDWFCESSKAFSSLQDLVQSTHSKRIQRLHDMPRKAGNILWWPFTQHKLVPEEIVTVIDSRCGENFAVHKVRNDREMIVRQFDACASWWTQGPDFALQIELARDMGYSAARYGHVMFPENVYEPALHCAELLLEGVGKGWASRTYYSDNGSTAIEIALKMAFRKFLLDNEMLMDSGNGISSERCIELRVLALNGSYHGDTLGAMEAQAPSSYTGFLQQPWYSGRGLFLDPPTIFIHKEIWNLSVPESFESDQLKPEDMRFSSSDALFCPSRDSSAIAGIYSAYISNQLLQFSKLSCSTQIGALIIEPVIQAAGGMHMIDPLFQRILVRECRNRRIPVVFDEVFTGFWRLGKESAAELLGCVPDVACFGKLMSGGIIPLAVTLTTEAVFEAFKGDSKLMALLHGHSYSAHAMGCTAAAKSIQWFRDPFTNPNVEFKEKKLKELWEMKLVLQISSLPAVQRVITLGTLCAVELKAEGSDAGYASLYASLLVQQLREDGLYVRPLGNVIYLMCGPCTSSHFCSQQLQQVYQRLHEFSQHQLRDSQSFQREG

SEQ ID NO: 9 - protein, 849 aa, Ostreococcus tauri
MSAPIARRASSVARGRTRWLTSTSIERSREWFVRSLGSASDGAASGNGRALELSIASFAIWGANTGVGKTLASAGLARAATRRGTATTFLKPVQTGYPEDSDAGFVARQTKGNETAGEHAAVASGSTEGGGEGFAAGVTRAHTEFAWRRAAGPHLTAAEEGRPVSDEEIVRATRNYLREFAESAREGNARGFAVIETAGGVASPGPSGTLQCEILRALRLPAILIGDGRLGGISTTTSAYESLHARGYDVVGVLMADDGYANHEAVAKILPSGTKVFALPPLPPQGKADAWLEKSAGVFSEAFETAMNWHEKRIQALQKLPGEALEKIWWPFTQHATVEPDTVTVIDGRYGEDFEIYDQEKGRDGAIDLRFDGAASWWTQGVSAELQQELNSAAANAAGRYGHVMFPENVHQPSMDATNALLEGPGKGWGSRVFYSDNGSTATEVGLKMAFRKYYVDAGLVSKDGHQRAKDLINDGSCHLPQLRIIALDGSYHGDTLGAMDMQSPSVFTGPMQTPWYQPRGLFLQPPSLAIRNGEWTVALPPHGLLNDGEDRLFHDGKDLESNTTWKSKADAFDIGKRLDSPLANEYRQAVDKALERAEQDAKAGHMGPVGALMMEPVLHGAGGMVLIDPLFQNILMKVCRERKIPIVLDEVFAGIWRLGTEGAWEIIGEKPDISCYAKLLTGGLMPMSATVATADVFDVFYGPGKAQGLLHGHSYTAYPIGCAVASKALSLYKDPAKNPNLIVDASSKGDTPTAYLRELWDESKLKEISNKSSVRRVFGQGCVLAVELEVEGEGGYSSNAAKEVVLRLRPHSVQARPLGNVVYLMCAPTTPKATCDRLLNVLAEELSE

SEQ ID NO: 10 - protein, 852 aa, Helianthus annuus
MFHPPLLFTLSRHFLRRHHHRHLSTLSHTLSHPIYTIWSANTGLGKTLVSAGIAAASVASPPSKLVYIKPIQTGYPLDSDAAFVYRKIPQILHRRNPNLTLTASNQTLNVSIPAAKVKIGDNFSDSRNGFGEFGFCEEMKVGEGVGEGSELICKTIYAWNEAISPHLASEREGALVGDEEVLVLLKKCLEIGVGSEGQGSVLSLVETAGGVASPGSSGSLQCDLYRPFRLPSILVGDGKLGGISGTISAYESLTIRGYDVVAIVLEDHGLENEVPLLSYLRNRVPVLVLPSIPKDPLDNLMEWYDESQSVFDSLKGIMLSSYEKQMYRLHEMPKKAQEIFWWPFTQHKLVPQEKVTVIDSRCGENFAVHKVKDHDYITQMFDACASWWTQGPNAALQIKLAREMGYTAARYGHVMFPENVYEPALQCAELLLQGVGKGWASRVFFSDNGSTAIEIALKMAFRKFLADNKLLMDLPHYNTDESNIELKVVALNGSYHGDTLGAMDAQAPSPYTGFLQQPWYTGRGVFLDPPFVSMCDGVWKIAVPKKMHYEDVILEDTSFNSRDQVFNRSRDDSKLASIYSTYISQELLPSSGSTKCSYAGALIIEPVIQGAGGMLMVDPLFQRILVKECKNRKIPVIFDEVFTGYWRLGVESAAELLFCQPDIACFAKLMTGGMIPLAATLATNDVFESFVGDSKLNALLHGHSYSAHAVGCATACNSIKWFKDSQTNPNLVPGQNLLRELWDAELVRKISFHPAVQRVVSLGTLFALELKVEDNDAGYASLSGTSLLLKLREDGIYMRPLGNVIYMMCGPCTSHRVCLQMLEKLYMRLDEFKQEKIGTARDYDHDHRLPIA

SEQ ID NO: 11 - protein, 864 aa, Quercus robur
MYFLISLHRRGHRHLRHVKDLSTTAHSQLHVPLNHPTFLVWASNTSLGKTLVSTGLAASSLLNPTPKPRKFLYLKPIQTGFPSESDSSSVFHKLSRLSLLRRHNLPHFSLFSSNHILKASFPAANSALGQEISETPHSGMRDLGYYEEAKPEGDVGGGAASQLVCKTLYAWREAVSPHLAAERESGVVEDAAVLELLQKCLRFELEGGGEKGKETEIFCVVETAGGVASPGPSGSLQCDLYRPFRLPGILVGDGRLGGISGTISAYESLKLRGYDIAAVVFEDHGLVNEMPLLSYLRNRVPVLVLPSVPKDLSNDLMEWFDESRGVFDSLKEEMLSAYSKRMMRLQDMPKKAGEIFWWPFTQHQLVPEEAVTVIDSRHGENFAVFKAQNNEFITQQFDACASWWTQGPDTTLQTELARDMGYAAARFGHVMFPENVYEPALECAELLLEGVGKGWASRTFFSDNGSTAIEIALKMAFRKFSYDHGILSDGRKDNTASQRVKLMVLALQGSYHGDTLGAMEAQAPSSYTGFLQQPWYTGRGLFLDPPTVYMCNGVWKLSLPEGLHLEIPKLENKGFSSRDEIFHKIRDKSDLARIYSSYISEQLSQYSGSGGFYHIGALIMEPVILGAGGMHMIDPLFQRVLVNECRSKKIPVIFDEVFTGFWRLGRETAAELLGCVPDIACFAKLMTGGIIPLAATLATNAIFNSFIGESKLKALLHGHSYSGHAMGCTAAVKSIKWFKDPQTNLNITDEGRSLRELWDEDVVQQISSHPAVERVVALGTLFALELRAEGSNAGYASQYASSLLQNLRQDGIYTRPLGNVIYLMCGPCTSPEICSQLLVKLYKRLEDFDQFHQIREENSESYGG

SEQ ID NO: 12 - protein, 764 aa, Thraustotheca clavata
MLTLSMRMRPRTLMKMSFSTRAGLGQLGCLTSLDQSVPNAVPTSLHAPIVQVFGSNTDVGKTVIAAGLCRAALLSSKNATVGYIKPLQTGGDAMMDAEFLNRHAGNADNRLTSHTLFSWETAVSPHLAASLENKAVDDATVVAKLSKTLKQIQKEEKGSALTVVETAGGVCSPSASGKFQCDVYRPLRLPTVLVGDGCLGGISATMSALDSLLIRGYDVSCIVLIEQDNLSNGEAISERASELGIPIFSLDKLPPQPEPLDLWYENSTETFRSVHDTIASTHKARVARLQELPSIAKKALWWPFTQHKSNKGVLVIDSAYGDTFATYNQKNQAIEPMYDACASWWTQGVGHGNAKMALSLATAAGRYGHVMFPENAHEPAIALSEMLLKSVGKNWAGKVFFSDDGSTAVEIGIKMALRKYAVDHGLGMSFEGINDVLVLAQEGCYHGDTLGTMNIAEPSVYNKTQHPWYKPKALFVHPPSVKVASNGQMQVTWQNVNPTLVTHLPTLDDLFDISRDTSPLTQQYTEYVESLLDSVPSTTVVGALIVEPILIGAGGMVFVDPLFQRTMVRVCQQRKIPVIFDEVFSGFWRLGAESARDLLQVNPDIACYAKLLTGGAVPMSTTLATNEVFDAFYTDSKADALLHGHSFTANPVGCAAAVTALDMYKTIEKKNSTRVHWNVDQVAALAARPGIARAFQLGTVLVIELESTSRGYTDTTGQDVLVRLRNHGIYARSLGNVLYIMASPLSTPATCIKIFNTVVDNLFA

SEQ ID NO: 13 - protein, 788 aa, Schizosaccharomyces japonicus
MLAHILFGANTEVGKTIWSTALCYASHYLAHAPVEYIKPVGAGPVEEADYVHLAKFVPCKCTNIHQFEKAMSPHMSEGAMQTTDASIVSALRKHLQASSFKNTFVETAGGVSSPAPSGSLQCDLYRPLRLPVLLIGDSKLGGISTTIAAYEQLKVRGYDISAILMLRDPENAPQYENWKYFSEIWSKSYSCHDKENIKCFTIPSPPALSQDAEADFKAMQKYYSESASVGYDVLKHIEQVQKTRLARLDDMINQTEKHIWYPFTQHTLVNPKEIAVIDSAHGDMYTVFAPNAKRKSEEALADGSKKEGSVAVEENKYKELFDASASWWTQGLGHGDPYLAMSAAYAAGRYGHVLLPNCTNEPALGLAEDLLSTVGKGWANRVYYSDNGATGMEIALKMALKAARIQHQLSGKIEIIGLEGAYHGDTIGTMDACPPNVYNVEVDWYDGRGIWFDSPVVKVSNGEVSVQWEDKSFSFTALDDVYNVSSRVNSHLAKQYASFIGEKVRTAMQQGHQFGALVIEPLVVGAGGMLFVDPLFQRVLVDIAKGRLPESPWYTEAHENKKAGIPVVFDEVFIGLYRLGQLRSADILGVYPDVQVNAKLLTGGLVPLSVTLASEELFEYFLSDKKSDALLHGHSYTAHPVGCHVARQSLKRLDQLNRTAWTENKQRWLEFFGCQNVNGVLSSRNPNAWSVWDPEWVYEVSKLECVESIMALGSLASVVLKAGDHGYQSSAAGQYLSRLREFSTGAEEYGILARPLGNVVYFMTSQITPTHTVKEIQAKIIDVFSQPR

SEQ ID NO: 14 - protein, 765 aa, Gibberella zeae
MAPVPALLWRSLRTHQVYGANTDVGKTIFSTVLCNAASKKGDRTWFLKPVSTGAVDEADGCHIQRFASSTNHETLFQYDIPCSPHIAAKVSGKPIPSDEAVLAKIHDSVSRYASEGPGWLFLETAGGVHSPGPSGTPQADLYTPLRAPVILVGDSKLGGISQTISAYESLRMRGHDIESILLFQDMKYENYQYLKDYFSKQGGIPVDTVPEPPSRLPDIQQDTEQMREYYASQTSNVSHVLDHLDKRGKQRISRLESLSEKASKSIWYPFTQQKLVTADTISAIDSAHGDYFQVLNKTYENLLQPAFDGSASWWSQGLGHANSRLTLAAAYAAGRYGHVMFAEAIHEPALALAEMMLEGAQNSRFSRVFYSDNGSTGCEVAVKMALRAARLRYGWGPNDNLQILGLKGSYHGDTIGAMDCAEPCVYNEKIEWYEGKGYWFDYPTIQCVEGKWVVSAPNGMGQDQKLESISEVFNLESRLKTEQYRGYEEYIEKTLKKLQDQGRKFGALMMEPIILGAGGMIFVDPLFQRALVDVVRRSPHLFGTKDALPAQGPQWSGLPVIFDEVFTGLYRLGRFTAASFLGTEADISVNAKLLTGGLVPLCTTMASESIFDAFKSDDKSDALLHGHSYTAHAVGCQVAVESVSEMQSMEKNGEWEWAEKDWAEDETQAWSVWSRDFVTDMSHNPQVLGVWALGSVLAISLRDDDGVGYKSLAAKKIQSHLRQGTGSWNAHSRVLGNVFYVMASQKTSRQSIRELQGLLVDALSK

SEQ ID NO: 15 - protein, 827 aa, Hordeum vulgare
MLRRLLLRHARRLHYSSSFSSALPLSSPTFAVFGANTGVGKTLVSAGLAASLLRSHAASPSAVLYLKPLQTGFPDDSDAGFLYRRVPALLRPSHAAHLAASIDTLSPAPSVETQSPSREEAAFSYDGEGEAVRKLLACRTLYAWREAVSPHLAAEREGMAVEDAHVRSLLAACLVEGENLGKGEGDVWKLVETAGGVASPGPSGTLQCDLYRQFRLPTILVGDGRLGGISCTLSAYETLLLRGYDVSAVILEDHGLSNNKFLLSYLRNRVPVLVLPPIPEDPSDDLTDWFSESSSVFGLLEDALRSFHLTRIQRLNSMQRKSKDLLWWPFTQHNLVPVDCVTVIDSRCGESFSAYKVKDNKMMMIPQFDACASWWTQGPDSKLQIELARDMGYAAARYGHVMFPENAHEPALRCAELLLGGIGKDWASRVYYSDNGSTAIEIALKMAFRKFSLDRGILLDSDKSIITNGRNIQLKVLALKGSYHGDTLGAMEAQAPSAYTSFLQQPWYSGRGLFLDPPTVFIKNGTCTLSLPQAIQNCHLSPGDKCFPSLAEVFCKTRDSSSAADLYSTYISQQLSEYSLSSNTEHIAALIIEPVIQGAGGMHMIDPLFQRVLVHECRDRKIPVIFDEVFTGFWRLGVESASELLGCLPDVACYAKLMTGGLVPLAATVTTEAVFEAFKSDSKLTALLHGHSYTAHAMGCSAAVKAIQWFRDPSTNSNLDFDCMKLKELWDGTLVNQLSSLPNVKRIVSLGTLCAIELQAEGSDAGYASLYARSLIQQLREEDDIYVRPLGNVIYLMCGPCTTRDVCTEQLSKVYRRISHFNPVHRG

SEQ ID NO: 16 - protein, 838 aa, Brassica napus
MSPVTATLLRHRLRHLRHHHIRLNSTAVPSHFNLPLNHPTYLIWSANTSLGKTLVSTGIASSFLLHQPSSSPPAAHSAKLLYLKPIQTGFPSDSDSRFVFSKLDSLSLRRRIPLSVSNSVLRSSLPVAESMRRNIKVSESGMCDLNFREEKTVTGAPELLCKTLYAWEAAISPHLAAEREHATVEDSVVLKMVEQEMECGSKANVLCLVETAGGVASPGPSGTLQCDLYRPFRLPGILVGDGRLGGISGTIAAYESLKLRGYDVAAVVFEDHGLVNEVPLTSYLRNKVPVLVLPPVPKDPSDDLIEWFVESDGVFKALKEVMVSVYLERVERLNGMAKQAGEVFWWPFTQHKLVPEDNVTIIDSRCGENFSVFKASDNNSITQQFDACASWWTQGPDPAFQAELAREMGYTAARFGHVMFPENVYEPALKCAELLLDGVGKGWASRVYFSDNGSTAIEIGLKMAFRKFCVDHETLFGLSEDAEEKKHVDVKVLALRGSYHGDTLGAMEAQAPSPYTGFLQQPWYTGRGLFLEPPTVFLSNGTWNLSLPESFSQTASEECGTFTTRDEIFDKSRDTSVLATVYSTYVSEQLQEYSGISQSAHVGALIIEPVIHGAGGMHMVDPLFQRVLVNECRNRKIPVIFDEVFTGFWRLGVETTADLLGCKPDIACFAKLLTGGMIPLAVTLATDAVFDSFSGDSKLKALLHGHSYSAHAMGCATATKAIEWFKDPETNHNIDSQRGTLRELWDEELVQHISCHCAVERVVVLGTLFALELKVDASNSGYASLYAKSLLEMLREDGIFMRPLGNVVYLMCGPCTSPEICRRLLTKLHKRLGEFNRA

SEQ ID NO: 17 - protein, 858 aa, Gossypium hirsutum
MLLRRHHHHRLFRLRLLLLHHHHHQFQSFSTLTIPLSHPTYIIWSSNTSLGKTLVSTGLSSSFLLSPSSSSKKFLYLKPLQTGFPSDSDSRFLFQKLSSLSLSRNLPLFSSHSVLLSSLPAAKSFKPNEFSLNESREMCELGFYEEKKVLEAGRVAPELVSETIYAWEGALSPHFAAEREGGAVEDSELVKRLETRLKEGLLEGGVERGKLDGFCVVETAGGVASPGPSGTLQCDLYRPLRFPGVLVGDGRLGGISGTISAYESLKLRGYDVVAIVIEDHGLINEIPLKSYLRSRVPVFVLPPIPQDPSNDLMEWFDESCNVFNSLKDIMLEAYLERMRRLNEMPRKAGDVFWWPFTQHKLVPPSTVTVIDSRCGENFSVYKVQNKEYITQQFDACASWWTQGPDATLQTELARDMGYAAARFGHVMFPENVYEPALECAELLLDGVGKGWATRVYFSDNGSTAIEIALKMAFRKFSSDHGILPELLKNNPTESCTELMVLALKGSYHGDTLGAMEAQAPSSFTGFLQQPWYTGRGLFLDPPTVFMHNGKWTVSLPEVFHSATLKPEDTTFRSRDEIFLKSRDESNLAGLYSLYVSQQLSQYSTLSETNKQIGALIMEPIIQGAGGMHMVDPLFQRMLVNECRHCRIPVIFDEVFTGFWRLGVESAAELLGCVPDIACFAKLMTGGIIPLAATMATDAIFDSFTGDSKLKALLHGHSYSAHAMGCTAAAKSIKWFKDPNTNLNITSEKSQKMLLRELWDAELVQQFSSHPSISRVVTLGTLFALELQADESDAGYASLYARSLVQMLREDCIYTRPLGNVIYVMCGPCTSPKMCTLQLLKLYTKLEEFTLVKTKMNII

SEQ ID NO: 18 - protein, 821 aa, Oryza sativa
MLRLLRHARRHSTSSSSSAAAAAVPLTSPAFAVFGANTGVGKTLVSAGLVASLLASPSPSPSTVAYLKPLQTGFPDDSDARFVFDRAPALLRRLRLAGGGASTRLVASNHTLFPSPAVDPLPERQDTVVNYGGEEGVEEKALVCRTVYAWREPVSPHLAAEREGMPVEDEEVRWLVDRWLAEEDGGGEVWKVLETAGGVASPGPSGTLQCDLYRSSRLPAVLVGDGRLGGISSTLSAYETLLLRGYDVGSVILEDRGLSNDRFLLSYLRKRVPVHVLPPIPEDPKDDLTDWFSESSSAFSSLKDSLQSFHSRRVQRLNSMQRKSKYLLWWPFTQHDLVPVDSVTVIDSRFGENFSAYKVKDKTIVPQFDACASWWTQGPDSNLQIELARDMGYAAARYGHVMFPENVHEPALRCAELLLGGVGKDWASRVYFSDNGSTAIEIALKMAFRKYACDHGIIVDSEKDIRSEGSVHFKVLALNGSYHGDTLGAMEAQAPSAYTSFLQQPWYSGRGLFLDPPTVYIKNKSANLSLPPSIMHDQLSSCDTCFSSLTEVFCKTRDTSSAANVYVSYISQQLSQYAMSNNSEHIAALIIEPVIQGAGGMHLIDPLFQRLLVKECKNRKIPVIFDEVFTGFWRLGVESASELLGCFPDISCYAKLMTGGIVPLAATLATEPIFEAFRSDSKLTALLHGHSYTAHPMGCTAAVKAIQWYKDPSTNSNIDLDRMKLKELWDSALVNHLSSLPNVKRVVSLGTLCAIELKAEGSDAGYASLYASSLIRQLREEDNIYARPLGNVIYLMCGPCTTQDSCTRQLAKVHRRLQKLN

SEQ ID NO: 19 - protein, 821 aa, Glycine max
MFRFPSVLLSRRLHRRELSSSTTSHPPSLPLSHPIYLIWGSNTGVGKTLVSAGIAAAFLLSSPTPSQFHYLKPLQTGFPSDSDSRFVFNKLRQLSLIRNPHIPLSASHRVINVSPAVTASNPLAGEEGVGPPPELLCKTLYAWEEAVSPHLAAEREGLVVKDSAVLETLGECLEDVAECGAGKERSEVLCVVETAGGVASPGPSGSLQCDLYRPFRIPAVLVGDGRLGGISGTISAYESLKLRGYDVVAVVFEDHGLLNEGPLMSYMRNKVPVLVLPPIPKDPLNDLMEWFESSHYIFSNLKEIMLSAYFERIKKLHDMPREARDIIWWPFTQHKLVPDGGVTVIDSRCGENFSIFKVQKTEVIAPQFDACASWWTQGPDAIMQTELAREMGYAAARFGHVMFPENVHEPALNCAELLLQGVGKGWASRTYFSDNGSTAIEIALKMAFRKFSVDHGLIVHCHEDTTNERSTELMVLALQRSYHGDTLGAMEAQAPSSYTGFLQQPWYTGRGLFLDPPSVFMHNSTWNVSIPEGYHLESLKGKSITFASPDEIFHKGRDKSELATVYSSYISKVLSGFRGSNNVGALIMEPVIQGAGGMHMVDPLFQRVLVNECRSRKIPVIFDEVFTGFWRLGVETAVELIHCVPDIACFGKLLTGGIIPLAVTLATNAVFDSFIGDSKLKALLHGHSYSAHAMGCAAAVKSIQWFKDPCSNPNITSEGRLLRELWDDKMVHRISSHPAIQRVVALGTLCALELKAEGNNAGYGSLYARPLLEKLREDGVYMRPLGNVIYLLCGPCTSPEVCNQLLVKLLRRLEEFDVGKN

SEQ ID NO: 20 - protein, 824 aa, Triticum aestivum
MLRRLLLRQARRRHCSSSFSSALPLSSPTFAVFGANTGVGKTLVSAGLAASLLRSHAASPSAVLYLKPLQTGFPADSDAGFLYRRVPALVRPSHAAHLVASIDTLSPAPSVETPSPSREEAAFSYDGEGKAVRKLLACRTLYAWREAVSPHLAAEREGMTVEDAHVRSLLAACLAEGETLGKGEGDVWKLVETAGGVASPGPSGTLQCDLYRHFRLPIILVGDGRLGGISGTLSAYETLLLRGYDVSAVILEDCGLSNDKFLLSYLRNRVPVLVLPPIPEDPSDDLTDWFSESSSVFGLLEDALRSFHLKRIQRLNSMQRKSKDLLWWPFTQHNLVPVDCVTVIDSRCGESFSAYKVKDNKLMMIPQFDACASWWTQGPDSKLQIELARDMGYAAARYGHVMFPENAHEPALRCAELLLGGIGKDWASRVYYSDNGSTAIEIALKMAFRKFSLDRGILLDSDKSITNERNIQLKVLALKGSYHGDTLGAMEAQAPSAYTSFLQQPWYSGRGLFLDPPTVFIRNGTCALSLPQSIQNCHLSPGDKCFPSFAEVFCKSRDSSAAADLYSTYISQQLSEYSMSSNTEHIAALIIEPVIQGAGGMHMIDPLFQRVLVHECRDRKIPVIFDEVFTGFWRLGVESASELLGCLPDVACYAKLMTGGIVPLAATVTTEAVFEAFKSDSKLTALLHGHSYTAHAMGCSAAVKAIQWFRDPSTNSNLDFDCMKLKELWDGTLVNQLSSLPNVKRVVSLGTLCAIELQAEGSDAGYASLYARSLIQQLREEDDIYVRPLGNVIYLMCGPCTTRDVCTEQLSKVYRRISHFNPIH

SEQ ID NO: 21 - protein, 812 aa, Arabidopsis thaliana
MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSSLPAAKSLGLNVEVSESGMCSLNFRDEKTVTGAPELLCKTLYAWEAAISPHLAAERENATVEDSVVLQMIEKCLKEEMECGVKSEKSDLLCLVETAGGVASPGPSGTLQCDLYRPFRLPGILVGDGRLGGISGTIAAYESLKLRGYDIAAVVFEDHGLVNEVPLTSYLRNKVPVLVLPPVPKDPSDDLIEWFVESDGVFKALKETMVLANLERLERLNGMAKLAGEVFWWPFTQHKLVHQETVTVIDSRCGENFSIYKASDNSSLSQQFDACASWWTQGPDPTFQAELAREMGYTAARFGHVMFPENVYEPALKCAELLLDGVGKGWASRVYFSDNGSTAIEIALKMAFRKFCVDHNFCEATEEEKHIVVKVIALRGSYHGDTLGAMEAQAPSPYTGFLQQPWYTGRGLFLDPPTVFLSNGSWNISLPESFSEIAPEYGTFTSRDEIFDKSRDASTLARIYSAYLSKHLQEHSGVRQSAHVGALIIEPVIHGAGGMHMVDPLFQRVLVNECRNRKIPVIFDEVFTGFWRLGVETTTELLGCKPDIACFAKLLTGGMVPLAVTLATDAVFDSFSGDSKLKALLHGHSYSAHAMGCATAAKAIQWFKDPETNHNITSQGKTLRELWDEELVQQISSHSAVQRVVVIGTLFALELKADASNSGYASLYAKSLLIMLREDGIFTRPLGNVIYLMCGPCTSPEICRRLLTKLYKRLGEFNRT

SEQ ID NO: 22 - protein, 712 aa, Zea mays
MQSPAVEAKAESLHESQEKVVTYGAGAAEQTKVLACCTAYAWREPVSPHLAAEREGMAAGDDEVKGCVEQWLLEEGIGEGGEVWKILETAGGVASPSASGTLQCDLYRPFRLPAILVGDGRLGGISSTLSAYETLLLRGYDVSAVILEDRELSNDKFLLSYLRNRVHVLILPQIPEDPLDDLTDWFSESSSIFIFLKDELQSFHSRRIERLNSMQRKSKALLWWPFTQHNLVPQDSVTVIDSRYGENFSVYKIKDKMLIPQFDACASWWTQGPDSNLQIELARDMGYAAARYGHVMFPENVHEPALHSAEVLLGGVGKGWASRVYYSDNGSTAIEIALKMAFRKFSLDHGIMASSENSTRNERNIQLKVLALNGSYHGDTLGAMEAQAPSAYTSFLQQPWYSGRGLFLDPPTVYIKSEVYNISLPQSMQHDPQTYGFFSQAEVFCKTRDKTFAADLYSSYIKQKLSEFSLSSSSEHLAALIIEPVIQGAGGMLMIDPLFQRVLVSECRSRKIPVIFDEVFTGFWRLGVESASELLGCLPDIACYAKLMTGGIIPLAATLATEEVFESFESDSKLTALLHGHSYTAHAMGCTAALKAMQWYRGPSTNSNLDTDHMKLKELWDGTLVKQLSSLPNVKRVVSLGTLCAIELKAEGADAGYASLYASSLVQQLRKEDDIYIRPLGNVIYLMCGPCTPRDSCTRQLLKVHRRLCALN

SEQ ID NO: 23 - protein, 786 aa, Taxus chinensis
MSRSGQAGEDRTPKQKLHIPSGFEVDLSHPTYVIWGSNTGVGKTLVSTGLAASLLNASQNLLYVKPVQTGFPQDSDSRYVSHKVKDILKQRKPSNNVHISHQVLKASPRAWELLPGSKGFGEVASYERAIQGMRNVCDYEGQTVQGLYKGLADLKLVCKTIYGWYEAVSPHLAAAGEKCSVDDCEVVENLQNSLREFNVDENNTNRWSIIETAGGVASPGPSGTLQCDLYRPFRLPSILVGDGRLGGISGTISAYETLCIRGYDICAVILADTGLSNEKCLSTYLHDRVPVFVLPKIPDDTNNTLLEWFSESYKVFSEIKDTLQTSYFKRIYRLHEMPMKARQILWWPFTQHSLVTEQSISLIDSRFGENFGVYKGKNHKDVKESIVQQFDACASWWTQGPSAILQTELAREVGYTTGRFGHVMYPENVYEPALHCAELLLEGVGKGWATRVFLSDNGSTAIEISLKMAFRKFAFDHGFLPEHFNEDLRKICPQFKVEIGTISYTTCAFFIEMTMEEFSEESYTGRGLFLDPPTVFLHNEEWQLHLPDALMYSDLKYPGLVIHASGGMHMIDPLFQRDLVKECRKRRIPVIFDEVFTGFWRLGAESAAEILGCAPDIACYAKLLTGGIVPLAATLATESIFEAFEGKSKLFALLHGHSYSGHAIGCAAAIKAIQWFKDAEMNLNICTDGSRLQELWDSKLVAKISALSSVKRVIALGTICALELQADESNTGYGSLLASSLVQKLRDDGVYMRPLGNVIYLMCGPATPPAMCSTLLQKLHKRLDEC

SEQ ID NO: 24 - protein, 878 aa, Physcomitrium patens
MSSHPSPIEDADEARCVAENVADALDVSYPTYMVWGSNTGVGKTLVSAGLCSAILRLSKCQGNSGAKDLLYLKPVQTGFPSDSDARYVYQEVSRVGRLHPEAISEGLFLCNHTLQVSPAVQISARAGASVPVQPQSAWNAGGLSSSLNGTGMKDFCSYESQLLQGVKSRNIPADGDVYGRAQLRNTLSRFTCKTMWAWYEPISPHLAAAREGSTVPDTMVVEAIQRSLQALSSSRKTSGHREDAGDCAEKWAILETAGGVASPGPSGSLQCDMYRSLRFSGLLVGDGRLGGISTTIAAYESLRSRGYDIPAIVIVDSGLSNEEPVLQYLRHKIPVVVLPPLPEDPNKRLEEWFRLAEGTFLELMNVLENSHSKRLRRLEEMPKMAGEILWWPFTQHDLVAQDSITLIDSRSGENFSVYKSGTPSTGKTIATQFDACASWWTQGPDSILQPEIAREVAYTAGRYGHVMFPENVHEPALRCAEILLAGAGRGWAERVFFSDNGSTAIEVAIKMAFRKYIVDHGLSDPSTIERRGGQLKVVALKGSYHGDTLGAMEAQAPSAYTGFKQQPWHVGRGFFLEPPTVFQRQGRWHLQLPRVFHDSDSSDSASCWKSRDEIFDICRDSTPLANLYIRSINQQLAAAVREENNVPAALIIEPVMHGAGGMDMIDPLYQRMLVRECRRRGIPTIFDEVLSGCWRFGVESAVDLLGCSPDIACYSKLLSGGLVPLAATLATQAVFEAFRGISKLDALLHGHSYTAHAVGCASAVVSLQYFSDPSKNSNLLPGGRRLRELWKPELVSLISSHQSVERVVSVGTIFALELRANAAESGYASMLSKELVENLRQEGIYTRPLGNVVYLMCGPLTQPKTCTTMLEKLLALLG

SEQ ID NO: 25 - protein, 811 aa, Setaria italica
MSSAAATVPLSTPTFAVFGANTGVGKTLVSAGLVAALLNSRSPSVSAVSYLKPLQTGYPADSDARFVFARAPALLRASSSPRATRLVASCRTLFPSPAVGPEAEPLHQSQEKVVTYGGDGAEEETKVLACRTVYAWREPVSPHLAAEREGMAAGDDEVRGCVDQWLMEEGVGEGGEVWKVLETAGGVASPSASGTLQCDLYRPFRLPAILVGDGRLGGISSTLSAYETLLLRGYDVSTVILEDRDLTNDTFLLSYLRNRVHVLALPQIPEDPSDDLTDWFSESSSVFNLLKDSLQSFHSKRVERLNGMQKKSKDLLWWPFTQHNLVPQDSVTVIDSRCGENFSAYKIKDNTMMLVPQFDACASWWTQGPDSDLQIELARDMGYAAARYGHVMFPENVHEPALRAAELLLGGVGKGWASRVYYSDNGSTAVEIALKMAFRKFSLDHGILADSEKSIRNERNIQLKVLALNGSYHGDTLGAMEAQAPSAYTSFIQQPWYSGRGLFLDPPTVCIKSETCNVSLPQSMQHYPRTSGDTCFPSLAEVFCKTRDTSSAADLYLSYIKQQLSEFSQSSNSDHLAALIIEPVIQGAGGMLMIDPLFQRVLVRECRSRKIPVIFDEVFTGFWRLGVESASELLGCLPDIACYAKLMTGGIVPLAATLATEEVFESFKSDSKLTALLHGHSYTAHAMGCMAALKAIQWYRDPSTNSNLDLDHMKLKELWDGALVKQLSSLPNVKRVVSLGTLCAIELKAEGSDAGYASLYASSLVQQLRREDDIYIRPLGNVIYLMCGPCTPRDSCSRQLHKVLRRLCDLN

SEQ ID NO: 26 - protein, 839 aa, Phoenix dactylifera
MPPELDLSHPTLVVWGSNTGVGKTIVSAGLAASVLSPSSPNTATFLYLKPVQTGFPRDSDSRFVYRKVSDLFRHRLGGFSAQLLASNHALKASVPAAREVLGARTERPGEEGEEASTGLCCYEETRVGTGESTTGDFKLVCKTLYGWKEAISPHLAVEREGMAVEDSSLREVLGRLLNSSMGGGGDRRKENVWRVIETAGGVASPGPSGSIQCDLYRPFRLPAVLVGDGHLGGISTTLSAYESLTLRGYDVIAIILADHGLSNEIPLLSNLRHRVPVLVLPPVPEDPLNDLVDWFCESSKAFSSLQDLVQSTHSKRIQRLHDMPRKAGNILWWPFTQHKLVPEEIVTVIDSRCGENFAVHKVRNDREMIVRQFDACASWWTQGPDFALQIELARDMGYSAARYGHVMFPENVYEPALHCAELLLEGVGKGWASRTYYSDNGSTAIEIALKMAFRKFLLDNEMLMDSGNGISSERCIELRVLALNGSYHGDTLGAMEAQAPSSYTGFLQQPWYSGRGLFLDPPTIFIHKEIWNLSVPESFESDQLKPEDMRFSSSDALFCPSRDSSAIAGIYSAYISNQLLQFSKLSCSTQIGALIIEPVIQAAGGMHMIDPLFQRILVRECRNRRIPVVFDEVFTGFWRLGKESAAELLGCVPDVACFGKLMSGGIIPLAVTLTTEAVFEAFKGDSKLMALLHGHSYSAHAMGCTAAAKSIQWFRDPFTNPNVEFKEKKLKELWEMKLVLQISSLPAVQRVITLGTLCAVELKAEGSDAGYASLYASLLVQQLREDGLYVRPLGNVIYLMCGPCTSSHFCSQQLQQVYQRLHEFSQHQLRDSQSFQREG

SEQ ID NO: 27 - protein, 815 aa, Ostreococcus tauri
MLGSASDGAASGNGRALELSIASFAIWGANTGVGKTLASAGLARAATRRGTATTFLKPVQTGYPEDSDAGFVARQTKGNETAGEHAAVASGSTEGGGEGFAAGVTRAHTEFAWRRAAGPHLTAAEEGRPVSDEEIVRATRNYLREFAESAREGNARGFAVIETAGGVASPGPSGTLQCEILRALRLPAILIGDGRLGGISTTTSAYESLHARGYDVVGVLMADDGYANHEAVAKILPSGTKVFALPPLPPQGKADAWLEKSAGVFSEAFETAMNWHEKRIQALQKLPGEALEKIWWPFTQHATVEPDTVTVIDGRYGEDFEIYDQEKGRDGAIDLRFDGAASWWTQGVSAELQQELNSAAANAAGRYGHVMFPENVHQPSMDATNALLEGPGKGWGSRVFYSDNGSTATEVGLKMAFRKYYVDAGLVSKDGHQRAKDLINDGSCHLPQLRIIALDGSYHGDTLGAMDMQSPSVFTGPMQTPWYQPRGLFLQPPSLAIRNGEWTVALPPHGLLNDGEDRLFHDGKDLESNTTWKSKADAFDIGKRLDSPLANEYRQAVDKALERAEQDAKAGHMGPVGALMMEPVLHGAGGMVLIDPLFQNILMKVCRERKIPIVLDEVFAGIWRLGTEGAWEIIGEKPDISCYAKLLTGGLMPMSATVATADVFDVFYGPGKAQGLLHGHSYTAYPIGCAVASKALSLYKDPAKNPNLIVDASSKGDTPTAYLRELWDESKLKEISNKSSVRRVFGQGCVLAVELEVEGEGGYSSNAAKEVVLRLRPHSVQARPLGNVVYLMCAPTTPKATCDRLLNVLAEELSE

SEQ ID NO: 28 - protein, 831 aa, Helianthus annuus
MLSTLSHTLSHPIYTIWSANTGLGKTLVSAGIAAASVASPPSKLVYIKPIQTGYPLDSDAAFVYRKIPQILHRRNPNLTLTASNQTLNVSIPAAKVKIGDNFSDSRNGFGEFGFCEEMKVGEGVGEGSELICKTIYAWNEAISPHLASEREGALVGDEEVLVLLKKCLEIGVGSEGQGSVLSLVETAGGVASPGSSGSLQCDLYRPFRLPSILVGDGKLGGISGTISAYESLTIRGYDVVAIVLEDHGLENEVPLLSYLRNRVPVLVLPSIPKDPLDNLMEWYDESQSVFDSLKGIMLSSYEKQMYRLHEMPKKAQEIFWWPFTQHKLVPQEKVTVIDSRCGENFAVHKVKDHDYITQMFDACASWWTQGPNAALQIKLAREMGYTAARYGHVMFPENVYEPALQCAELLLQGVGKGWASRVFFSDNGSTAIEIALKMAFRKFLADNKLLMDLPHYNTDESNIELKVVALNGSYHGDTLGAMDAQAPSPYTGFLQQPWYTGRGVFLDPPFVSMCDGVWKIAVPKKMHYEDVILEDTSFNSRDQVFNRSRDDSKLASIYSTYISQELLPSSGSTKCSYAGALIIEPVIQGAGGMLMVDPLFQRILVKECKNRKIPVIFDEVFTGYWRLGVESAAELLFCQPDIACFAKLMTGGMIPLAATLATNDVFESFVGDSKLNALLHGHSYSAHAVGCATACNSIKWFKDSQTNPNLVPGQNLLRELWDAELVRKISFHPAVQRVVSLGTLFALELKVEDNDAGYASLSGTSLLLKLREDGIYMRPLGNVIYMMCGPCTSHRVCLQMLEKLYMRLDEFKQEKIGTARDYDHDHRLPIA

SEQ ID NO: 29 - protein, 844 aa, Quercus robur
MSTTAHSQLHVPLNHPTFLVWASNTSLGKTLVSTGLAASSLLNPTPKPRKFLYLKPIQTGFPSESDSSSVFHKLSRLSLLRRHNLPHFSLFSSNHILKASFPAANSALGQEISETPHSGMRDLGYYEEAKPEGDVGGGAASQLVCKTLYAWREAVSPHLAAERESGVVEDAAVLELLQKCLRFELEGGGEKGKETEIFCVVETAGGVASPGPSGSLQCDLYRPFRLPGILVGDGRLGGISGTISAYESLKLRGYDIAAVVFEDHGLVNEMPLLSYLRNRVPVLVLPSVPKDLSNDLMEWFDESRGVFDSLKEEMLSAYSKRMMRLQDMPKKAGEIFWWPFTQHQLVPEEAVTVIDSRHGENFAVFKAQNNEFITQQFDACASWWTQGPDTTLQTELARDMGYAAARFGHVMFPENVYEPALECAELLLEGVGKGWASRTFFSDNGSTAIEIALKMAFRKFSYDHGILSDGRKDNTASQRVKLMVLALQGSYHGDTLGAMEAQAPSSYTGFLQQPWYTGRGLFLDPPTVYMCNGVWKLSLPEGLHLEIPKLENKGFSSRDEIFHKIRDKSDLARIYSSYISEQLSQYSGSGGFYHIGALIMEPVILGAGGMHMIDPLFQRVLVNECRSKKIPVIFDEVFTGFWRLGRETAAELLGCVPDIACFAKLMTGGIIPLAATLATNAIFNSFIGESKLKALLHGHSYSGHAMGCTAAVKSIKWFKDPQTNLNITDEGRSLRELWDEDVVQQISSHPAVERVVALGTLFALELRAEGSNAGYASQYASSLLQNLRQDGIYTRPLGNVIYLMCGPCTSPEICSQLLVKLYKRLEDFDQFHQIREENSESYGG

SEQ ID NO: 30 - protein, 734 aa, Thraustotheca clavata
MSLDQSVPNAVPTSLHAPIVQVFGSNTDVGKTVIAAGLCRAALLSSKNATVGYIKPLQTGGDAMMDAEFLNRHAGNADNRLTSHTLFSWETAVSPHLAASLENKAVDDATVVAKLSKTLKQIQKEEKGSALTVVETAGGVCSPSASGKFQCDVYRPLRLPTVLVGDGCLGGISATMSALDSLLIRGYDVSCIVLIEQDNLSNGEAISERASELGIPIFSLDKLPPQPEPLDLWYENSTETFRSVHDTIASTHKARVARLQELPSIAKKALWWPFTQHKSNKGVLVIDSAYGDTFATYNQKNQAIEPMYDACASWWTQGVGHGNAKMALSLATAAGRYGHVMFPENAHEPAIALSEMLLKSVGKNWAGKVFFSDDGSTAVEIGIKMALRKYAVDHGLGMSFEGINDVLVLAQEGCYHGDTLGTMNIAEPSVYNKTQHPWYKPKALFVHPPSVKVASNGQMQVTWQNVNPTLVTHLPTLDDLFDISRDTSPLTQQYTEYVESLLDSVPSTTVVGALIVEPILIGAGGMVFVDPLFQRTMVRVCQQRKIPVIFDEVFSGFWRLGAESARDLLQVNPDIACYAKLLTGGAVPMSTTLATNEVFDAFYTDSKADALLHGHSFTANPVGCAAAVTALDMYKTIEKKNSTRVHWNVDQVAALAARPGIARAFQLGTVLVIELESTSRGYTDTTGQDVLVRLRNHGIYARSLGNVLYIMASPLSTPATCIKIFNTVVDNLFA

SEQ ID NO: 31 - protein, 733 aa, Gibberella zeae
MCNAASKKGDRTWFLKPVSTGAVDEADGCHIQRFASSTNHETLFQYDIPCSPHIAAKVSGKPIPSDEAVLAKIHDSVSRYASEGPGWLFLETAGGVHSPGPSGTPQADLYTPLRAPVILVGDSKLGGISQTISAYESLRMRGHDIESILLFQDMKYENYQYLKDYFSKQGGIPVDTVPEPPSRLPDIQQDTEQMREYYASQTSNVSHVLDHLDKRGKQRISRLESLSEKASKSIWYPFTQQKLVTADTISAIDSAHGDYFQVLNKTYENLLQPAFDGSASWWSQGLGHANSRLTLAAAYAAGRYGHVMFAEAIHEPALALAEMMLEGAQNSRFSRVFYSDNGSTGCEVAVKMALRAARLRYGWGPNDNLQILGLKGSYHGDTIGAMDCAEPCVYNEKIEWYEGKGYWFDYPTIQCVEGKWVVSAPNGMGQDQKLESISEVFNLESRLKTEQYRGYEEYIEKTLKKLQDQGRKFGALMMEPIILGAGGMIFVDPLFQRALVDVVRRSPHLFGTKDALPAQGPQWSGLPVIFDEVFTGLYRLGRFTAASFLGTEADISVNAKLLTGGLVPLCTTMASESIFDAFKSDDKSDALLHGHSYTAHAVGCQVAVESVSEMQSMEKNGEWEWAEKDWAEDETQAWSVWSRDFVTDMSHNPQVLGVWALGSVLAISLRDDDGVGYKSLAAKKIQSHLRQGTGSWNAHSRVLGNVFYVMASQKTSRQSIRELQGLLVDALSK

SEQ ID NO: 32 - protein, 813 aa, Hordeum vulgare
MSSSFSSALPLSSPTFAVFGANTGVGKTLVSAGLAASLLRSHAASPSAVLYLKPLQTGFPDDSDAGFLYRRVPALLRPSHAAHLAASIDTLSPAPSVETQSPSREEAAFSYDGEGEAVRKLLACRTLYAWREAVSPHLAAEREGMAVEDAHVRSLLAACLVEGENLGKGEGDVWKLVETAGGVASPGPSGTLQCDLYRQFRLPTILVGDGRLGGISCTLSAYETLLLRGYDVSAVILEDHGLSNNKFLLSYLRNRVPVLVLPPIPEDPSDDLTDWFSESSSVFGLLEDALRSFHLTRIQRLNSMQRKSKDLLWWPFTQHNLVPVDCVTVIDSRCGESFSAYKVKDNKMMMIPQFDACASWWTQGPDSKLQIELARDMGYAAARYGHVMFPENAHEPALRCAELLLGGIGKDWASRVYYSDNGSTAIEIALKMAFRKFSLDRGILLDSDKSIITNGRNIQLKVLALKGSYHGDTLGAMEAQAPSAYTSFLQQPWYSGRGLFLDPPTVFIKNGTCTLSLPQAIQNCHLSPGDKCFPSLAEVFCKTRDSSSAADLYSTYISQQLSEYSLSSNTEHIAALIIEPVIQGAGGMHMIDPLFQRVLVHECRDRKIPVIFDEVFTGFWRLGVESASELLGCLPDVACYAKLMTGGLVPLAATVTTEAVFEAFKSDSKLTALLHGHSYTAHAMGCSAAVKAIQWFRDPSTNSNLDFDCMKLKELWDGTLVNQLSSLPNVKRIVSLGTLCAIELQAEGSDAGYASLYARSLIQQLREEDDIYVRPLGNVIYLMCGPCTTRDVCTEQLSKVYRRISHFNPVHRG

SEQ ID NO: 33 - protein, 815 aa, Brassica napus
MSTAVPSHFNLPLNHPTYLIWSANTSLGKTLVSTGIASSFLLHQPSSSPPAAHSAKLLYLKPIQTGFPSDSDSRFVFSKLDSLSLRRRIPLSVSNSVLRSSLPVAESMRRNIKVSESGMCDLNFREEKTVTGAPELLCKTLYAWEAAISPHLAAEREHATVEDSVVLKMVEQEMECGSKANVLCLVETAGGVASPGPSGTLQCDLYRPFRLPGILVGDGRLGGISGTIAAYESLKLRGYDVAAVVFEDHGLVNEVPLTSYLRNKVPVLVLPPVPKDPSDDLIEWFVESDGVFKALKEVMVSVYLERVERLNGMAKQAGEVFWWPFTQHKLVPEDNVTIIDSRCGENFSVFKASDNNSITQQFDACASWWTQGPDPAFQAELAREMGYTAARFGHVMFPENVYEPALKCAELLLDGVGKGWASRVYFSDNGSTAIEIGLKMAFRKFCVDHETLFGLSEDAEEKKHVDVKVLALRGSYHGDTLGAMEAQAPSPYTGFLQQPWYTGRGLFLEPPTVFLSNGTWNLSLPESFSQTASEECGTFTTRDEIFDKSRDTSVLATVYSTYVSEQLQEYSGISQSAHVGALIIEPVIHGAGGMHMVDPLFQRVLVNECRNRKIPVIFDEVFTGFWRLGVETTADLLGCKPDIACFAKLLTGGMIPLAVTLATDAVFDSFSGDSKLKALLHGHSYSAHAMGCATATKAIEWFKDPETNHNIDSQRGTLRELWDEELVQHISCHCAVERVVVLGTLFALELKVDASNSGYASLYAKSLLEMLREDGIFMRPLGNVVYLMCGPCTSPEICRRLLTKLHKRLGEFNRA

SEQ ID NO: 34 - protein, 830 aa, Gossypium hirsutum
MSTLTIPLSHPTYIIWSSNTSLGKTLVSTGLSSSFLLSPSSSSKKFLYLKPLQTGFPSDSDSRFLFQKLSSLSLSRNLPLFSSHSVLLSSLPAAKSFKPNEFSLNESREMCELGFYEEKKVLEAGRVAPELVSETIYAWEGALSPHFAAEREGGAVEDSELVKRLETRLKEGLLEGGVERGKLDGFCVVETAGGVASPGPSGTLQCDLYRPLRFPGVLVGDGRLGGISGTISAYESLKLRGYDVVAIVIEDHGLINEIPLKSYLRSRVPVFVLPPIPQDPSNDLMEWFDESCNVFNSLKDIMLEAYLERMRRLNEMPRKAGDVFWWPFTQHKLVPPSTVTVIDSRCGENFSVYKVQNKEYITQQFDACASWWTQGPDATLQTELARDMGYAAARFGHVMFPENVYEPALECAELLLDGVGKGWATRVYFSDNGSTAIEIALKMAFRKFSSDHGILPELLKNNPTESCTELMVLALKGSYHGDTLGAMEAQAPSSFTGFLQQPWYTGRGLFLDPPTVFMHNGKWTVSLPEVFHSATLKPEDTTFRSRDEIFLKSRDESNLAGLYSLYVSQQLSQYSTLSETNKQIGALIMEPIIQGAGGMHMVDPLFQRMLVNECRHCRIPVIFDEVFTGFWRLGVESAAELLGCVPDIACFAKLMTGGIIPLAATMATDAIFDSFTGDSKLKALLHGHSYSAHAMGCTAAAKSIKWFKDPNTNLNITSEKSQKMLLRELWDAELVQQFSSHPSISRVVTLGTLFALELQADESDAGYASLYARSLVQMLREDCIYTRPLGNVIYVMCGPCTSPKMCTLQLLKLYTKLEEFTLVKTKMNII

SEQ ID NO: 35 - protein, 811 aa, Oryza sativa
MSTSSSSSAAAAAVPLTSPAFAVFGANTGVGKTLVSAGLVASLLASPSPSPSTVAYLKPLQTGFPDDSDARFVFDRAPALLRRLRLAGGGASTRLVASNHTLFPSPAVDPLPERQDTVVNYGGEEGVEEKALVCRTVYAWREPVSPHLAAEREGMPVEDEEVRWLVDRWLAEEDGGGEVWKVLETAGGVASPGPSGTLQCDLYRSSRLPAVLVGDGRLGGISSTLSAYETLLLRGYDVGSVILEDRGLSNDRFLLSYLRKRVPVHVLPPIPEDPKDDLTDWFSESSSAFSSLKDSLQSFHSRRVQRLNSMQRKSKYLLWWPFTQHDLVPVDSVTVIDSRFGENFSAYKVKDKTIVPQFDACASWWTQGPDSNLQIELARDMGYAAARYGHVMFPENVHEPALRCAELLLGGVGKDWASRVYFSDNGSTAIEIALKMAFRKYACDHGIIVDSEKDIRSEGSVHFKVLALNGSYHGDTLGAMEAQAPSAYTSFLQQPWYSGRGLFLDPPTVYIKNKSANLSLPPSIMHDQLSSCDTCFSSLTEVFCKTRDTSSAANVYVSYISQQLSQYAMSNNSEHIAALIIEPVIQGAGGMHLIDPLFQRLLVKECKNRKIPVIFDEVFTGFWRLGVESASELLGCFPDISCYAKLMTGGIVPLAATLATEPIFEAFRSDSKLTALLHGHSYTAHPMGCTAAVKAIQWYKDPSTNSNIDLDRMKLKELWDSALVNHLSSLPNVKRVVSLGTLCAIELKAEGSDAGYASLYASSLIRQLREEDNIYARPLGNVIYLMCGPCTTQDSCTRQLAKVHRRLQKLN

SEQ ID NO: 36 - protein, 804 aa, Glycine max
MSSSTTSHPPSLPLSHPIYLIWGSNTGVGKTLVSAGIAAAFLLSSPTPSQFHYLKPLQTGFPSDSDSRFVFNKLRQLSLIRNPHIPLSASHRVINVSPAVTASNPLAGEEGVGPPPELLCKTLYAWEEAVSPHLAAEREGLVVKDSAVLETLGECLEDVAECGAGKERSEVLCVVETAGGVASPGPSGSLQCDLYRPFRIPAVLVGDGRLGGISGTISAYESLKLRGYDVVAVVFEDHGLLNEGPLMSYMRNKVPVLVLPPIPKDPLNDLMEWFESSHYIFSNLKEIMLSAYFERIKKLHDMPREARDIIWWPFTQHKLVPDGGVTVIDSRCGENFSIFKVQKTEVIAPQFDACASWWTQGPDAIMQTELAREMGYAAARFGHVMFPENVHEPALNCAELLLQGVGKGWASRTYFSDNGSTAIEIALKMAFRKFSVDHGLIVHCHEDTTNERSTELMVLALQRSYHGDTLGAMEAQAPSSYTGFLQQPWYTGRGLFLDPPSVFMHNSTWNVSIPEGYHLESLKGKSITFASPDEIFHKGRDKSELATVYSSYISKVLSGFRGSNNVGALIMEPVIQGAGGMHMVDPLFQRVLVNECRSRKIPVIFDEVFTGFWRLGVETAVELIHCVPDIACFGKLLTGGIIPLAVTLATNAVFDSFIGDSKLKALLHGHSYSAHAMGCAAAVKSIQWFKDPCSNPNITSEGRLLRELWDDKMVHRISSHPAIQRVVALGTLCALELKAEGNNAGYGSLYARPLLEKLREDGVYMRPLGNVIYLLCGPCTSPEVCNQLLVKLLRRLEEFDVGKN

SEQ ID NO: 37 - protein, 801 aa, Triticum aestivum
MLSSPTFAVFGANTGVGKTLVSAGLAASLLRSHAASPSAVLYLKPLQTGFPADSDAGFLYRRVPALVRPSHAAHLVASIDTLSPAPSVETPSPSREEAAFSYDGEGKAVRKLLACRTLYAWREAVSPHLAAEREGMTVEDAHVRSLLAACLAEGETLGKGEGDVWKLVETAGGVASPGPSGTLQCDLYRHFRLPIILVGDGRLGGISGTLSAYETLLLRGYDVSAVILEDCGLSNDKFLLSYLRNRVPVLVLPPIPEDPSDDLTDWFSESSSVFGLLEDALRSFHLKRIQRLNSMQRKSKDLLWWPFTQHNLVPVDCVTVIDSRCGESFSAYKVKDNKLMMIPQFDACASWWTQGPDSKLQIELARDMGYAAARYGHVMFPENAHEPALRCAELLLGGIGKDWASRVYYSDNGSTAIEIALKMAFRKFSLDRGILLDSDKSITNERNIQLKVLALKGSYHGDTLGAMEAQAPSAYTSFLQQPWYSGRGLFLDPPTVFIRNGTCALSLPQSIQNCHLSPGDKCFPSFAEVFCKSRDSSAAADLYSTYISQQLSEYSMSSNTEHIAALIIEPVIQGAGGMHMIDPLFQRVLVHECRDRKIPVIFDEVFTGFWRLGVESASELLGCLPDVACYAKLMTGGIVPLAATVTTEAVFEAFKSDSKLTALLHGHSYTAHAMGCSAAVKAIQWFRDPSTNSNLDFDCMKLKELWDGTLVNQLSSLPNVKRVVSLGTLCAIELQAEGSDAGYASLYARSLIQQLREEDDIYVRPLGNVIYLMCGPCTTRDVCTEQLSKVYRRISHFNPIH

SEQ ID NO: 38 - DNA, 7742 bp, synthetic construct
  source 1..7742: mol_type = other DNA
ggcagcagccatcatcatcatcatcacagcagcggcctggtgccgcgcggcagccatatgaagtccaccagcgtgtccccgtttcacttacctctgaatcaccctacgtacctgatttggagtgctaacacaagcctcggtaagactctcgtctctaccggcatcgcggcaagcttccttttgcagcagccgtcaagtagtgcgactaaactgctctacctcaagccaattcagactggcttcccttcagactcagactcccgcttcgtgttttcaaagttagactcattatccctgcgtcgccaaatcccaatttctatctcaaacagcgtgttgcattcatcactgccagccgctaaatcccttgggctgaacgtagaagttagtgagtctggcatgtgctcccttaactttcgcgacgaaaagacagttacgggcgcgcctgagctgctgtgtaagacgctttacgcatgggaagcagcgatctccccgcatttagcggccgagcgcgaaaatgctacagtcgaggactctgttgtcctgcaaatgatcgagaagtgcctcaaagaggagatggagtgtggagttaagagcgagaagagtgatctgttgtgtcttgtagaaactgctggcggcgtggcgagccctggcccgtccggtacgttacaatgtgacttataccgtccctttcgtctcccaggaatcttggtcggggacggccgtctgggcggtatcagcggaacaattgcggcatacgaatcgctgaagcttcgtggttacgatatcgcggcagtcgtgtttgaagatcacggcctcgtgaacgaggtgccactgacttcatacttacgtaacaaggtcccagtgttagtcttaccgcctgtcccaaaggacccgtcggacgatctcatcgagtggtttgtcgagtcagacggtgtatttaaggcgctgaaggagacgatggtattagctaacttagagcgtctggagcgtcttaacggtatggcgaagctggcgggtgaggtgttttggtggccgtttacacaacataagttagtccatcaagagaccgtaaccgtaattgacagtcgttgcggcgagaacttttcgatctataaggccagcgataatagttccttatctcaacagtttgatgcgtgtgcatcgtggtggacacaggggcccgaccctacttttcaagcggaactggcacgtgaaatgggctatacagctgctcgttttggtcatgtcatgtttcccgaaaatgtatacgagccagcgttaaagtgcgccgagttgctcttggacggagtagggaagggttgggcatcgcgcgtatacttttctgacaatggcagtacggctatcgaaatcgccttaaagatggcattccgtaagttctgtgtcgatcacaacttttgcgaggcaacggaagaggaaaagcacattgtcgtgaaggtgatcgcccttcgcgggtcctaccatggggataccttgggcgcgatggaagcgcaggctccgagtccttataccggtttccttcaacaaccttggtatactggccgtgggttattcttggacccgccgacggtgtttctctctaatggtagttggaatatttccttaccagagtcgtttagcgagattgcacctgaatacgggacgttcactagtcgtgacgaaatctttgacaagagccgtgacgcgagcacgttagcccgcatctacagcgcgtatctgagcaagcacttgcaggaacacagtggtgtgcgccaaagtgcgcacgtcggtgccttgattatcgagcccgtcatccacggggcgggcggaatgcacatggtggacccgttattccagcgtgtcctcgtgaatgaatgccgtaatcgtaagatcccggtcatctttgacgaggtatttacagggttttggcgtcttggggttgagacgacgaccgagttgcttggctgtaagccggatattgcgtgttttgccaaactcttaactggtggaatggtacctttagcagttacgctggcgacagacgcagtgttcgactctttctccggcgatagcaagttaaaggcactccttcacggacatagctacagcgcgcacgcaatgggctgcgccaccgccgccaaggccatccaatggttcaaggacccggagactaatcataacattacctcgcagggtaaaactttacgcgagttatgggacgaggaactcgttcagcaaattagtagccacagtgcggtccaacgcgttgtggttatcggaacgttatttgcgctcgagcttaaggcagacgcgtctaacagtggctacgcctctctttatgcgaaaagcctgctgattatgttgcgcgaggacggtatctttacccgccccttaggtaatgtcatctaccttatgtgcggaccttgtaccagcccagagatctgccgccgcctcttgaccaagctttataagcgtctgggtgagtttaaccgcacctaagcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatg